(19) United States
(12) Patent Application Publication
(10) Pub. No.: US 2014/0330196 A1
(43) Pub. Date: Nov. 6, 2014

(54) APPARATUS FOR STIMULATING HAIR GROWTH AND/OR PREVENTING HAIR LOSS

(71) Applicant: PILOGICS L.P., Haifa (IL)

(72) Inventors: Dov Ingman, Haifa (IL); Erez Manor, Herzlia (IL)

(21) Appl. No.: 14/361,742

(22) PCT Filed: Dec. 6, 2012
(86) PCT No.: PCT/IB2012/057041

§ 371 Date: May 30, 2014

Related U.S. Application Data
(60) Provisional application No. 61/568,202, filed on Dec. 8, 2011.

(51) Int. Cl. A61M 37/00; A61M 5/46; A61N 5/06

(52) U.S. Cl. CPC A61M 37/0015; A61N 5/0617; A61M 5/46; A61M 2037/0023; A61M 2037/0061; A61M 2037/0007; A61N 2005/063; A61N 2005/0642
USPC 604/21; 604/22; 607/92; 604/117; 604/506

(57) ABSTRACT

A device to stimulate a scalp comprising an array of stimulating elements adapted to pierce the skin of the scalp no deeper than a thickness of a dermis, wherein the stimulating elements are arranged along a circumference of at least one wheel and the wheel is adapted to roll over the scalp.

### RELATED APPLICATION

The present application is a PCT Application which claims the benefit of priority of U.S. Provisional Application No. 61/568,202, filed 8 Dec. 2011, the contents of which are incorporated herein by reference in their entirety.

### FIELD AND BACKGROUND OF THE INVENTION

The present invention, in some embodiments thereof, relates to a device and method for stimulating skin and, more particularly, but not exclusively, to a device and method for directly stimulating the skin below the surface of the scalp to promote hair growth.

McDonough et al, in US patent application 2006/0253079 disclose “The present invention features a stratum corneum-piercing device including a microprotrusion member having a skin-contacting surface and plurality of stratum corneum piercing microprotrusions thereon, the device being adapted to move the microprotrusion member lateral to the surface of the skin surface upon contact with the skin.”

Laubach, H.-J. T. (2006, February). Skin responses to fractional photothermolysis. Lasers in Surgery and Medicine. Lasers in Surgery and Medicine, 38(2), 142-149, disclose “A single treatment with fractional photothermolysis induces a wound healing response in the dermis.”

Mayumi Ito, Z. Y. (2007, May). Wnt-dependent de novo hair follicle regeneration in adult mouse skin after wounding. Nature (447), 316-320, discloses “Here we show that, after wounding, hair follicles form de novo in genetically normal adult mice.”

Chuong, C.-M. (2007, May). Regenerative biology: New hair from healing wounds. Nature (447), 265-266, discloses “It now seems that, as they heal, open skin wounds in adult mice form new hair follicles that follow similar developmental paths to those of embryos.”

Sugimoto Y, L.-S. I.-T. (1995, May). Cations inhibit specifically type I 5 alpha-reductase found in human skin. J Invest Dermatol (104(5)), 775-778, discloses “The results showed that type I 5a-reductase was strongly inhibited by Cd, Cu, and Zn and moderately inhibited by Ni and Fe . . . . The data showed that cations could specifically control 5a-reductase activity expression, which is more strongly inhibited in a target tissue, especially the skin.”

Additional background art includes:

Hiroyuki Hori, G. M. (1972). The Thickness of Human Scalp: Normal and Bald, Journal of Investigative Dermatology (58), 396-399.

Groux et al, in U.S. Pat. No. 5,800,477 disclose “Hair growth method and apparatus”.

Whang et al, in U.S. Pat. No. 7,559,944 disclose “Hair growth apparatus”.

Kim et al, in US patent application 2007/0038275 disclose “High-frequency electrotherapy apparatus”.

Pitzen et al, in U.S. Pat. No. 6,834,206 disclose “Method for the electrical stimulation of human tissue to encourage hair growth”.

Hans-Joachim et al, Skin Responses to Fractional Photothermolysis, Lasers in Surgery and Medicine 38:142-149 (2006), disclose “A single treatment with fractional photothermolysis induces a wound healing response in the dermis.”

Nisato et al in U.S. Pat. No. 8,048,019 disclose “Multiple Nozzle Transdermal Drug Delivery System.”

Chizmadzhev et al, “Electrical Properties of Skin at Moderate Voltages: Contribution of Appendageal Macropores,” Biophysical Journal Volume 74 February 1998 843-856.

Maulsby et al, The interrelationship between the galvanic skin response, basal resistance, and temperature, Journal of Comparative and Physiological Psychology, Vol 53(5), October 1960, 475-479.

Taberner et al, Needle-free jet injection using real-time controlled linear Lorentz-force actuators, Medical Engineering & Physics—November 2012 (Vol. 34, Issue 9, Pages 1228-1235).

Rhodes W., Shallow Dermal Delivery of Vaccines Using Jet Injectors, Drug Development and Delivery, Vol. 3 No. 1 January/February 2003. Grimnes, Pathways of Ionic Flow through Human Skin in vivo, Department of Biomedical Engineering, Acta Derm Venereol, (Stockh) 1984; 64: 93-98.

### SUMMARY OF THE INVENTION

An aspect of some embodiments of the invention relates to an apparatus and a method for selectively applying at least one stimulation modality to the skin in-situ. In an exemplary embodiment of the invention, stimulation is applied directly to at least one deeper layer of the skin of the scalp. Optionally, mechanical stimulation is applied, for example by at least one wheel comprising an array of needles. Optionally or alternatively, vibrational stimulation is applied, for example by vibration of the needle wheel. Alternatively or additionally, thermal stimulation is applied, for example by the needles. Alternatively or additionally, ions having 5-alpha-reductase inhibiting properties are directly deposited below the skin surface, for example by the needles.

In some embodiments, light stimulation is applied, for example using optical fibers to direct light to under the skin.

According to an aspect of some embodiments of the present invention, there is provided a device to stimulate a scalp comprising an array of stimulating elements adapted to pierce skin of said scalp no deeper than a thickness of a dermis, wherein the stimulating elements are arranged along a circumference of at least one wheel, wherein the wheel is adapted to roll over said scalp.

According to some embodiments of the invention, the stimulating elements comprise needles.

According to some embodiments of the invention, the stimulating elements release ions.

According to some embodiments of the invention, the stimulating elements are further adapted to additionally stimulate the scalp to enhance movement of the ions to a level sufficient to cause a biological effect.

According to some embodiments of the invention, a first group of stimulating elements of the array comprises a first metal; a second group of stimulating elements of the array comprises a second metal and the electrochemical gradient is sufficient to cause ions to travel in an amount sufficient to cause a biological effect.

According to some embodiments of the invention, the stimulating elements of the arrays comprise at least one metal and the metal comprises sufficient polarity and sufficient power with suitable polarity to cause ion injection.

According to some embodiments of the invention, the ions comprise at least one of copper ions and zinc ions.

According to some embodiments of the invention, the stimulating elements are arranged to part hair on the scalp during piercing by the stimulating elements; the arranged to part hair comprises stimulating elements separated from one another along a dimension to form a gap sufficiently wide to avoid trapping hairs; and the dimension comprises between 2 mm and 5 mm.

According to some embodiments of the invention, the stimulating elements of the array are separated by 0.1 mm to 1 mm along an axis; the stimulating elements are adapted to pierce skin to at least one depth between 100 micrometers and 1700 micrometers and the stimulating elements have a shape that forms a wound with a cross sectional area between 0.00001 mm² and 0.1 mm² at the dermis.

According to some embodiments of the invention, the device further comprises at least one element to vibrate at least one stimulating element.

According to some embodiments of the invention, vibrate comprises vibrating to increase a cross section of a wound under the scalp by the stimulating element by a factor ranging from 2×-20×.

According to some embodiments of the invention, vibrate comprises vibrating at a frequency ranging from 50 Hz-120 Hz and at an amplitude of 0.05 mm to 0.2 mm.

According to some embodiments of the invention, at least one stimulating element is electrically coupled to a power source.

According to some embodiments of the invention, the device further comprises ion injecting electrodes that touch the scalp connected to one terminal of the power source and an electrode that does not touch the scalp connected to a second terminal of the power source.

According to some embodiments of the invention, at least one stimulating element is configured to provide heat adapted to maintain a temperature of the stimulating elements during the piercing of the scalp.

According to some embodiments of the invention, the device further comprises a controller for regulating the application of at least one of: temperature of the stimulating elements, number of the piercings of the skin by the stimulating elements, vibration of the stimulating elements, and application of electrical current by the stimulating elements to the skin.

According to some embodiments of the invention, the device further comprises a memory, the memory coupled to the controller, the memory containing data correlating stimulation parameters with a treatment and a sensor, the sensor coupled to the controller, the sensor configured to monitor the piercing of the skin by the stimulating elements.

According to some embodiments of the invention, the device further comprises a drug reservoir comprising at least one drug to administer to the scalp.

According to some embodiments of the invention, the device further comprises at least one of a motor and a handle, each configured to displace the array across the scalp.

According to some embodiments of the invention, the device further comprises an encoder operable to count revolutions or partial revolutions of the wheel.

According to some embodiments of the invention, the stimulation comprises light.

According to some embodiments of the invention, the light is delivered through at least one of transparent discs, needles and optical fibers.

According to some embodiments of the invention, the stimulating elements comprise optical fibers acting as needles to pierce skin.

According to some embodiments of the invention, the stimulating elements comprise at least one injector configured to deliver stimulation directly into skin without needles.

According to an aspect of some embodiments of the present invention, there is provided a device to stimulate a scalp comprising an array of stimulating elements adapted to pierce skin of the scalp no deeper than a thickness of a dermis.

According to some embodiments of the invention, the stimulating elements are independently displaceable along a long axis of the stimulating elements to pierce the scalp in synchronized motion.

According to some embodiments of the invention, the stimulating elements comprise needles and the device further comprises a power source in electrical communication with at least two of the needles, the power source coupled to apply a voltage across the at least two needles; a vibrational element coupled to the array, the vibrational element operable to vibrate the needles along at least one axis; and a heat source thermally coupled to the needles, the heat source operable to raise needles to a temperature sufficient to raise a temperature of a volume of skin to within a range of 45-70 degrees Celsius.

According to some embodiments of the invention, at least one of the needles is coated or made from a first material that discharges zinc ions and at least one of the needles is coated or made from a second material that discharges copper ions.

According to an aspect of some embodiments of the present invention, there is provided a method of stimulating a scalp comprising forming channels at least below an epidermal layer of skin of the scalp; providing at least one stimulation from inside the channels; effecting tissue adjacent to the channels; and controlling the providing.

According to some embodiments of the invention, stimulating comprises wounding the skin in a non-contiguous pattern.

According to some embodiments of the invention, the wounding comprises wounding the skin at a density of 5-10 wounds per mm².

According to some embodiments of the invention, the controlling comprises wounding the skin in a period of time ranging from 0.01 seconds to 0.1 seconds per wound, to reduce a pain level.

According to some embodiments of the invention, the wounding comprises sufficiently wounding the skin to induce a wound healing response that regenerates hair follicles.

According to some embodiments of the invention, the wounding comprises selectively wounding at a depth selected according to a stage of baldness.

According to some embodiments of the invention, the stimulation is provided at multiple depths in the epidermis and/or dermis, ranging between 100 micrometers and 1700 micrometers.

According to some embodiments of the invention, the stimulating comprises applying a vibration under the skin.

According to some embodiments of the invention, the controlling comprises applying a vibration to increase the cross sectional size of a wound under the skin by a factor of 2-20×.

According to some embodiments of the invention, the stimulating comprises applying light under the skin.

According to some embodiments of the invention, the stimulating comprises applying heat sufficiently to induce a wound healing response that increases collagen production.

According to some embodiments of the invention, the controlling comprises applying sufficient heat to raise a temperature of a volume of skin to within a range of 45-70 degrees Celsius.

According to some embodiments of the invention, the stimulating comprises applying at least one voltage gradient to an area of the skin.

According to some embodiments of the invention, the stimulating comprises applying voltage in an opposite polarity to release copper ions under the skin.

According to some embodiments of the invention, the stimulation comprises forming a galvanic current that releases zinc ions under the skin.

According to some embodiments of the invention, the stimulating comprises applying an alternating current to alternate deposition of copper ions and zinc ions under the skin and a waveform of the alternating current is selected according to the ratio of the desired deposition of copper ions and zinc ions.

According to some embodiments of the invention, the stimulating comprises depositing a selected amount of at least one of copper ions and zinc ions under the skin to inhibit type I 5-alpha-reductase to stimulate hair growth; the selected amount of zinc ions ranges from 0.001 to 1 nanogram/cm² per treatment; the maximum total weekly dosage is between 2 nanograms/cm2 and 4 nanograms/cm2; and the selected amount of the copper ions ranges from 1% to 50% of the selected zinc ion amount.

According to some embodiments of the invention, controlling comprises applying the stimulation according to a position on the scalp and adjusting the providing according to hair growth.

According to some embodiments of the invention, controlling comprises measuring an impedance to determine contact of at least one needle with the skin.

According to some embodiments of the invention, a treatment session of the stimulating the scalp is repeated at least once daily and controlling comprises applying the stimulation during a particular treatment session to an area on the scalp smaller than the entire area being treated.

According to some embodiments of the invention, hair loss is treated by the stimulation of the scalp and the method further comprises comparing the providing to a treatment plan.

According to some embodiments of the invention, the stimulating comprises depositing at least one of copper ions and zinc ions under the skin to enrich the scalp with mineral nutrients in an amount sufficient to cause a biological effect; and create electrical fields.

An aspect of some embodiments of the invention relates to a kit for stimulating hair growth on a scalp comprising at least one wheel having an array of needles arranged along a circumference of the wheel, the wheel adapted for replacement in an apparatus for stimulating hair growth; and a power source for providing power to the apparatus.

In an exemplary embodiment of the invention, the kit further comprises a device for stimulating hair growth on a scalp, the device adapted to couple to the wheel.

In an exemplary embodiment of the invention, the kit further comprises software for communicating with a controller of the device for at least one of programming and monitoring a treatment of the device on the scalp.

In an exemplary embodiment of the invention, the kit further comprises an additive for applying to a scalp by a subject.

An aspect of some embodiments of the invention relates to a device to stimulate a scalp comprising an array of needles to pierce skin of the scalp no deeper than a thickness of a dermis, the needles are independently displaceable along a long axis of the needles.

In an exemplary embodiment of the invention, at least some needles are displaced along the long axis to pierce the scalp in synchronized motion.

An aspect of some embodiments of the invention relates to a device to stimulate a scalp comprising:
a. An array of needles adapted to piece skin of the scalp no deeper than a thickness of a dermis;
b. a power source in electrical communication with at least two of the needles, the power source coupled to apply a voltage across the at least two needles;
c. a vibrational element coupled to the array, the vibrational element operable to vibrate the needles along at least one axis; and
d. a heat source thermally coupled to the needles, the heat source operable to raise needles to a temperature.

In an exemplary embodiment of the invention, at least one of the needles is coated or made from a first material that discharges zinc ions and at least one of the needles is coated or made from a second material that discharges copper ions. Optionally, the needles are arranged along a circumference of a wheel adapted to roll over the scalp.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings and/or images. With specific reference now to the drawings and/or images in detail, it is stressed that the particulars shown are by way of example and for purposes of illustrative discussion of embodiments of the invention. In this regard, the description taken with the drawings and/or images makes apparent to those skilled in the art how embodiments of the invention may be practiced.

In the drawings:

FIG. 1A is an illustration of a device to stimulate hair growth, in accordance with an exemplary embodiment of the invention;

FIGS. 1B and 1C are illustrations of the function of the device, in accordance with an exemplary embodiment of the invention;

FIG. 1D is a face-on illustration of the device of FIG. 1A, in accordance with an exemplary embodiment of the invention;

FIG. 2 is a block diagram of the device of FIG. 1, in accordance with an exemplary embodiment of the invention;

FIGS. 3A-3C are images and illustrations of a prototype device to stimulate hair growth, useful in practicing some embodiments of the invention;

FIG. 4 is a flow chart of a method of stimulating hair growth, in accordance with an exemplary embodiment of the invention;

FIG. 5 is a flowchart of a detailed method of FIG. 4, in accordance with an exemplary embodiment of the invention;

FIGS. 6A-6C are illustrations of embodiments of needles and/or needle arrays, in accordance with some embodiments of the invention;

FIGS. 7A-7I are illustrations of embodiments of needle actuators used with needle arrays, in accordance with some embodiments of the invention;

FIGS. 8A-8D are illustrations of embodiments of depositing ions below the skin surface, in accordance with some embodiments of the invention;

FIG. 9 is an illustration of the device of FIG. 1 further comprising a mechanism for delivering drugs to below the skin surface, in accordance with some embodiments of the invention;

FIGS. 10A-10G are illustrations of embodiments of the hair stimulation device, in accordance with some embodiments of the invention;

FIG. 11 is an illustration of a monitoring and/or a feedback set-up used with the hair stimulation device, in accordance with some embodiments of the invention;

FIG. 12 is an illustration of a hair stimulation kit 1200 in accordance with some embodiments of the invention;

FIGS. 13A-13B are images of a scalp before and after hair stimulation treatment, useful in practicing some embodiments of the invention;

FIGS. 14A-14B are illustrations of embodiments of discs, in accordance with some embodiments of the invention;

FIG. 15 is an illustration of a light source in accordance with some embodiments of the invention; and

FIG. 16 is a bar chart summarizing experimental results achieved using methods in accordance with some embodiments of the invention.

### DESCRIPTION OF SPECIFIC EMBODIMENTS OF THE INVENTION

The present invention, in some embodiments thereof, relates to a device and method for stimulating skin and, more particularly, but not exclusively, to a device and method for directly stimulating the skin below the surface of the scalp.

An aspect of some embodiments of the invention relates to a device and a method for stimulating the skin below the surface, for example to stimulate hair growth. Optionally, the skin is the skin of the scalp. Optionally, the stimulation is directly delivered to the skin, at least to one or more layers below the surface, for example, by needles in-situ. Optionally, at least one stimulation modality is applied to the skin.

In an exemplary embodiment of the invention, stimulating hair growth comprises stimulating new hair growth. Alternatively or additionally, stimulating comprises maintaining the current amount of hair. Alternatively or additionally, stimulating comprises reducing the rate of hair loss. Alternatively or additionally, stimulating comprises enhancing implant success.

In an exemplary embodiment of the invention, the skin below the surface is stimulated by at least one needle, for example the needle piercing the skin and stimulating the skin in-situ below the surface. Optionally, an array of needles is used.

In an exemplary embodiment of the invention, locally delivered ions provide stimulation to the skin. Optionally, the movement of the ions is enhanced during and/or after their delivery beneath the skin. Optionally, the scalp is stimulated to enhance movement of the ions to a level sufficient to cause a biological effect. For example, the scalp is stimulated to enhance movement of the ions to a level exceeding 0.01 nanograms/cm². For example, an electric field is applied to cause the ions to move more quickly and/or greater distances. Optionally, light, heat and/or another means is applied to cause the ions to move more quickly and/or greater distances. Optionally, the stimulation causes increased blood flow and/or opening of capillaries.

In an exemplary embodiment of the invention, the needles provide mechanical stimulation to the skin, for example by traumatizing and/or wounding the skin. Optionally, the pattern of stimulation is selectable and/or controllable, for example manually by a user and/or automatically by a robot and/or by a user under automatic monitoring and/or guidance.

In an exemplary embodiment of the invention, the needles are shaped to part and/or displace exiting hair on the scalp, for example to allow the needle to penetrate the skin of the scalp covered hair. Optionally, the hairs are pushed into one or more sufficiently wide gaps between the needles. The width of the gap is, for example, at least 2 mm, at least 3 mm, at least 4 mm, or other smaller, intermediate or larger sizes are used. The height of the gap is, for example, at least 2 mm, at least 4 mm, at least 6 mm, at least 10 mm, or other smaller, intermediate or larger heights are used. Non-limiting examples of the arrangement of gaps and/or needles include; bull's eye, clover leaf, alternating rows, checkerboard pattern. Alternatively or additionally, needles and/or groups of needles are individually displaceable along the long axis of the needle, for example, to adjust to a variation in skin surface, for example according to the presence of hairs.

In an exemplary embodiment of the invention, the needles are relatively close together, for example, along one or more regions and/or axes of the array. Needles are spaced, for example, about 0.1 mm apart, about 0.5 mm apart, about 1 mm apart, or other smaller, intermediate or larger distances are used.

In an exemplary embodiment of the invention, the needles penetrate no deeper than the dermis. Optionally, the length of the needle is selected according to the estimated dermis thickness associated with a stage of baldness (e.g., before balding begins, initial stage, advanced stage), for example, as described by Hiroyuki et al, incorporated herein by reference. Needles length is selected to form a wound, for example, about 100 micrometers in depth, about 300 micrometers, about 500 micrometers, about 1000 micrometers, about 1400 micrometers, about 1700 micrometers, or other smaller, intermediate or larger values are used.

In an exemplary embodiment of the invention, the needles are relatively thin. For example, the cross sectional area of the needles forms a wound in the dermis having a cross sectional area of, for example, about 0.00001 mm², about 0.0001 mm², about 0.001 mm², about 0.01 mm², about 0.1 mm² about 1 mm², or other smaller, intermediate or larger values are used.

In an exemplary embodiment of the invention, the needles are inserted into the skin for a relatively short period of time, for example, about 0.01 seconds, about 0.05 seconds, about 0.1 seconds, or other smaller, intermediate or larger values are used.

In an exemplary embodiment of the invention, the array of needles is displaced over the scalp. Optionally, the array is displaced automatically, for example by a motor. Alternatively or additionally, the array is displaced manually, for example by the user. Alternatively, the array of needles is static with respect to the scalp.

In an exemplary embodiment of the invention, stimulation comprises vibrational stimulation, for example vibration of the needles. Optionally, needles are vibrated individually and/or in groups.

In an exemplary embodiment of the invention, needles are vibrated along an axis parallel to the lateral displacement of the needles along the scalp, for example, substantially perpendicular to the long axis. Alternatively or additionally, needles are vibrated along an axis perpendicular to the lateral displacement of the needles. Alternatively or additionally, needles are vibrated in one or more axes, for example omnidirectional. Alternatively or additionally, needles can flex, for example, omnidirectionally.

In some embodiments, needles are vibrated to relatively increase the wound cross section below the surface. Needles are vibrated to increase the size of the wound, for example, to about 2× larger than the cross sectional area of the needles, about 5× larger, about 10× larger, about 20× larger, or other smaller, intermediate or larger dimensions are used.

In an exemplary embodiment of the invention, needles are vibrated along the long axis. Optionally, the peak to peak vibration amplitude comprises of inserting the tip of the needle into the deepest part of the skin and/or removing the needle entirely from the skin. Optionally or additionally, the needles are vibrated once the needles have pierced the skin.

In an exemplary embodiment of the invention, the stimulation comprises applying one or more drugs directly below the skin surface, for example metals to preferably inhibit type I 5-alpha-reductase. Non-limiting examples of metals include copper and/or zinc. Optionally, metals are in ionic form, for example cations.

In an exemplary embodiment of the invention, ions are discharged from the needles, for example during piercing of the skin. Optionally, needles are coupled to the metals, for example coated by the metal.

In an exemplary embodiment of the invention, ions are discharged from the needles by a galvanic cell set-up. Optionally, a power source is electrically connected to at least two needles, wherein each needle is coupled to a different metal (e.g., at least two different types of metals). Alternatively or additionally, the needles are electrically connected without the power source, for example with a conductive wire. Alternatively or additionally, the needles are not directly electrically connected outside the skin, but are connected through the skin itself, for example in a galvanic corrosion set-up.

In an exemplary embodiment of the invention, ions are discharged from the needles by a set-up that does not include a self-defined galvanic cell. Optionally, an electrical power source is electrically connected to at least one needle, wherein the at least one needle is coupled to a metal (e.g., at least one type of metal). For example, the metal comprises sufficient polarity and the power source comprises sufficient voltage to cause ion injection. Optionally, needles are attached to two different metals which are not bridged. For example, a power source separately drives Zn and Cu into the scalp. In some embodiments, the needle is electrically connected without the power source, for example with a conductive wire.

In some embodiments the stimulation is delivered below the skin via an injection without needles. For example, the stimulation is delivered via a jet injection, with the jets optionally formed and/or travel in the air outside the skin. Optionally, the stimulation delivered by injection without needles comprises a cream. Optionally, the stimulation delivered by injection without needles comprises ions. In an exemplary embodiment of the invention, ions are deposited below the scalp by jet injection carried out by at least one jet. For example, one jet injects Zn ions and a second jet injects Cu ions.

In some embodiments, jet injectors are used to speed the process of the delivery of stimulation, for example, ion deposition, optionally in addition to needles, optionally or alternatively with a plurality operating simultaneously, for example, at least 2, 4, 10, 20 or smaller or intermediate or greater number of needles. Optionally or alternatively, the jet injectors form an array of injectors. Optionally or alternatively, the array of jet injectors form an array of injectors comprises jet injectors located close to each other, for example, in rows, columns and/or other formations. Optionally or alternatively, jet injectors are used to inject other types of ions and/or materials, such as vitamins. Optionally, at least some jet contains a different solution from others. Optionally, each jet deposits one type of ions at a specific location.

In some embodiments, the material in the jet is heated to cause micro-burns. Optionally, ions are deposited below the scalp by direct injection using a hollow needle containing ionized solution. In this case, a slower jet speed may be used, for example, just fast enough to exit the needle at a desired volume, but not enough to overpenetrate the skin, if at all.

In some embodiments the stimulation is delivered below the skin via an injection without needles together with stimulation delivered by needles. For example, jet injection delivery augments needle delivery. For example, stimulation may be delivered by an array of jet injectors alternating with needles.

In an exemplary embodiment of the invention, the dose of ions is selectable, for example, per needle. Optionally, the type of ion deposited is selectable, for example by controlling the polarity of the voltage at the needle. Optionally or additionally, the approximate amount (e.g., number) of ions deposited (e.g., per needle) is selectable, for example by controlling and/or calculating the charge passing through the needle.

In an exemplary embodiment of the invention, the stimulation comprises selectively heating areas below the surface of the skin of the scalp. Optionally, heating is sufficient to wound the skin, for example by protein denaturation.

In an exemplary embodiment of the invention, areas of below the skin are stimulated by heated needles piercing the skin. Optionally, the heating by individual and/or groups of needles is selectable and/or controllable. Optionally, the volume of skin wounded around the needle is selectable and/or controllable.

In an exemplary embodiment of the invention, the skin below the surface is heated to a temperature sufficient to induce a wound healing response. For example, to about 50 or 45 degrees Celsius, about 55, about 60, about 70 degrees Celsius, or other smaller, intermediate or larger temperatures or subranges thereof are used.

In an exemplary embodiment of the invention, the temperature of the skin below the surface is maintained during heating, for example, by a sufficiently large heat source thermally coupled to the needles.

In an exemplary embodiment of the invention, the stimulation comprises selectively applying voltage gradients and/or currents below the skin surface by the needles. Optionally, a plurality of voltage and/or current patterns are applied. In some embodiments of the invention, voltage gradients (e.g., relative between two needles), for example, are less than 1 volt, less than 3 volt, less than 6 volt, or other smaller, intermediate or larger gradients are used. Optionally, one or more electrodes or a different part of the device (e.g., the handle) serve as reference 0 volt for the applied voltage gradients. In some embodiments of the invention, electrical currents (e.g., total current through all needles at any point in time) are, for example, less than 0.05 mA, less than 0.1 mA, less than 0.2 mA, or other smaller, intermediate or larger values are used. In some embodiments of the invention, the voltage and/or current is alternated at a frequency of, for example, about 10 Hz, about 100 Hz, about 500 Hz, about 1000 Hz, or other smaller, intermediate or larger values are used.

An aspect of some embodiments of the invention relates to a method of stimulating hair growth on a scalp. In an exemplary embodiment of the invention, the method comprises selectively traumatizing the skin below the surface of the scalp. Optionally, selectively traumatizing comprises a non-contiguous pattern of relatively small wounds. For example, about 1 wound per mm² of scalp, about 5 wounds per mm² of scalp, about 10 wounds per mm² of scalp, or other smaller, intermediate or larger numbers of wounds.

In an exemplary embodiment of the invention, the method further comprises selectively applying a vibration during selective traumatization of the skin.

In an exemplary embodiment of the invention, the method further comprises selectively heating the tissue inside and/or surrounding the wounds.

In an exemplary embodiment of the invention, the method further comprises selectively depositing drugs, for example type I 5-alpha-reductase (e.g., metallic ions of copper and/or zinc), in and/or around the wounds.

In an exemplary embodiment of the invention, the method further comprises selectively applying one or more patterns of current and/or voltage inside and/or between wounds.

In an exemplary embodiment of the invention, the method further comprises monitoring the applied treatment, for example by a user against the prescribed treatment. Optionally, the treatment protocol is adjusted according to the monitoring, for example, treatment is intensified, one or more treatment modalities are added and/or removed, treatment is reduced.

In an exemplary embodiment of the invention, one or more treatment protocols and/or needle dimensions and/or needle array arrangement are selected to reduce pain or be painless. Non-limiting examples include; sufficiently thin needles, sufficiently short needles, pricking the skin with the needle during a relatively short period of time. Alternatively, the pain level is tolerable by the patient. Optionally or additionally, bleeding does not occur.

A particular feature of some embodiments of the invention, relates to the ability to replace the array of needles. Optionally, replacement needs are sterile. Optionally or additionally, replacement needs are low cost.

A particular feature of some embodiments of the invention, relates to the ability that the device is easy to handle and/or easy to use at home. Optionally, one or more wheels comprising needles on the outer circumference, are rolled over the scalp. Optionally or additionally, the device is light weight. Optionally or additionally, the device is maneuverable by holding a handle.

A particular feature of some embodiments of the invention, relates to the ability to apply multiple stimulation modalities underneath the surface of the skin. Optionally, the modalities are applied simultaneously. Inventors hypothesize that the multiple stimulation modalities have a synergistic effect, for example, increasing blood flow to the scalp, forming collagen, and/or regenerating hair.

In some embodiments, a plurality of treatments (ion, vibration, light, electricity and/or heat or other methods of stimulation) provides synergistic benefits to the treatments, when performed, for example, at the same time and location. Optionally, a plurality of treatments provides more efficacious results than a series of identical individual treatments. In some cases, applying the same treatments in sequential form would not suffice to provide the minimal stimulation required for healing. In other cases, sequential treatments may require strong and/or long treatments to reach an effective level.

In some embodiments, the synergistic effects include enhanced delivery of stimulation. For example, a plurality of stimulations may improve blood flow. In some embodiments, the combination of treatments is selected so as to increase blood flow beyond that level of blood flow achieved by the use of individual treatments.

In some embodiments, stimulation by ions and by vibration provides synergistic effects. Optionally, stimulation by light and heat provide synergistic effects. Optionally, stimulation by ions, vibration, light, electricity and heat provide synergistic effects. Optionally, stimulation by ions and electricity provide synergistic effects. Optionally, stimulation by ions and light provide synergistic effects. Optionally, stimulation by ions and heat provide synergistic effects. Optionally, other combinations of ions, vibration, light, electricity and/or heat or other methods of stimulation provide synergistic effects.

In some embodiments, the synergistic effects are enhanced by performing a plurality of stimulations in localized areas. Optionally, the synergistic effects create a strong gradient of effects, so degree of total stimulation at one area is greater than degree at a nearby area, possibly causing biological effects in itself. Optionally, the localization of the treatment prevents the dilution of the stimulation effects which may be otherwise caused by “spreading” different stimulation methods over a large area.

Possibly, the localization of the treatment prevents a weakening of the stimulation effects caused by limitations of time, energy and/or other resources. For example, the localization of the treatment may prevent dilution of the stimulation effects which may be caused by a lack of sufficient strength in an electrical field and/or a lack of sufficiently strong heat shock.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary Device

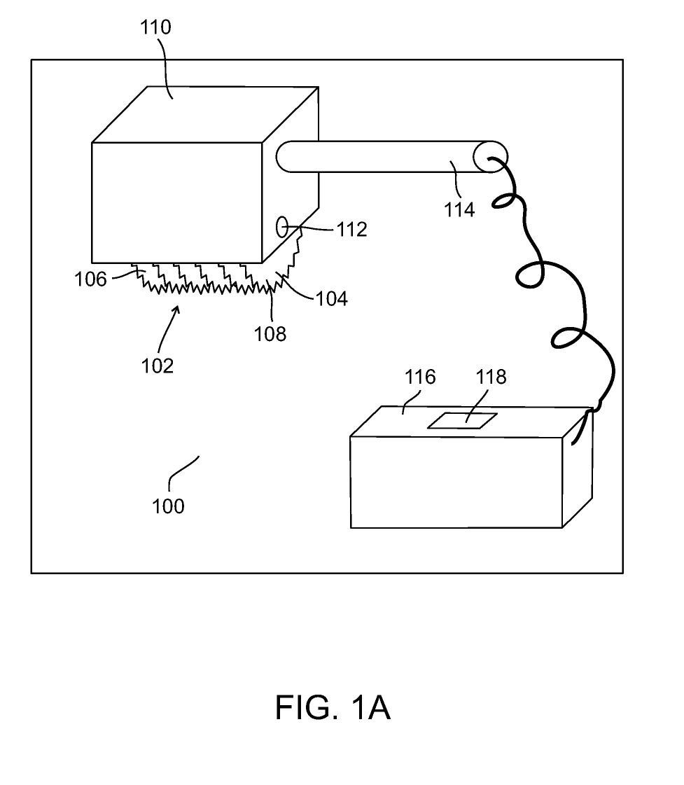

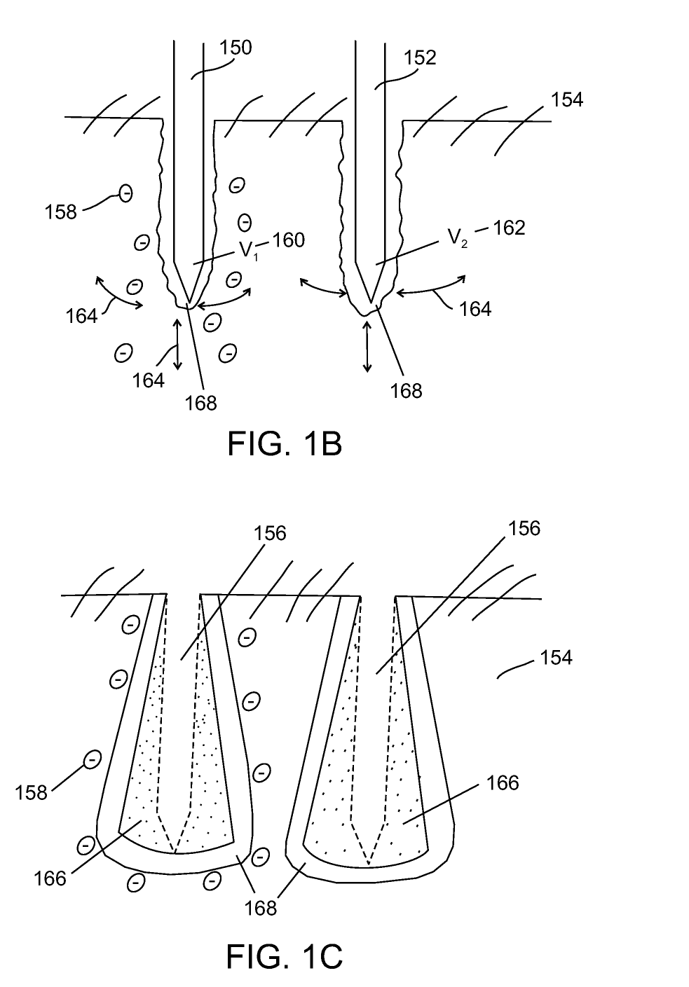

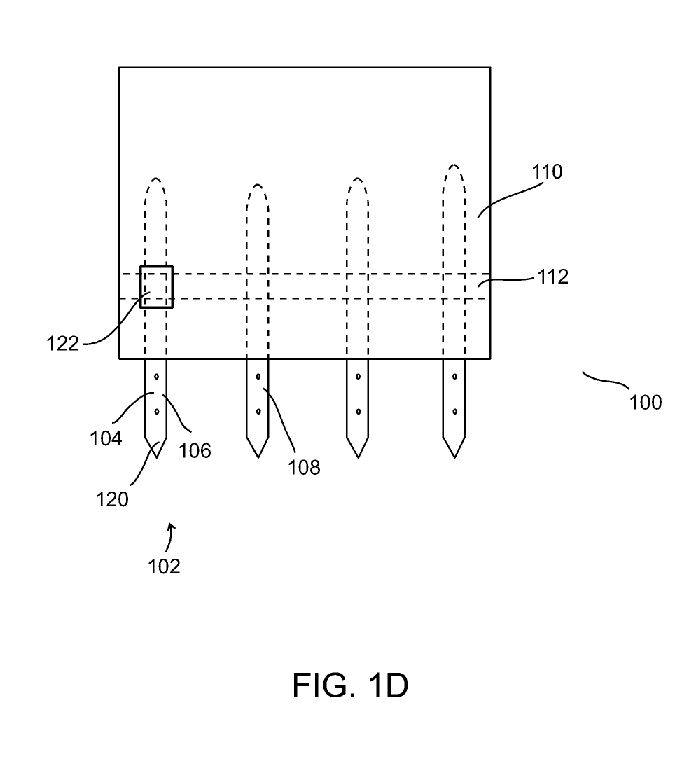

FIG. 1A is an illustration of an exemplary device 100 for promoting hair growth, in accordance with an exemplary embodiment of the invention. In an exemplary embodiment of the invention, device 100 applies one or more stimulatory modalities directly to the skin at least below the surface, for example by needles pricking the skin. Optionally, device 100 is used on the skin of the scalp of a patient.

In an exemplary embodiment of the invention, the depth of the needle penetration is controlled, for example, to reach different skin layers, for example, epidermis, dermis, depth of the hair follicle's bulb (e.g., a stem cell rich area), the depth of the hair follicle's papilla (e.g., rich capillary area). Optionally, different needles have different lengths. Alternatively or additionally, the depth of penetration is controlled for example by vibrating the needle. Optionally, different stimulations are applied at different depths and/or skin layers, either simultaneously and/or in parallel. Non-limiting examples include; discharging specific ions at specific targets, heating specific skin layers, causing greater microtrauma at different layers.

In an exemplary embodiment of the invention, needles 102 are arranged along the circumference of at least one disc 104, for example, 2, 4, 6, 8, or other smaller, intermediate or larger numbers of discs 104 are used. The diameter of discs 104 is, for example, about 2 cm, about 4 cm, about 6 cm, or other smaller, intermediate or larger diameters are used. The thickness of discs and/or needles is, for example, about 0.05 mm, about 0.1 mm, about 0.15 mm, or other smaller, intermediate or larger thickness are used.

A potential advantage of the disc design, is that the needle array is low cost and/or easy to replace. Another potential advantage of needles 102 arranged along the circumference of discs 104 is that device 100 can be rolled on the scalp, potentially providing for increased control and/or monitoring of needles 102 piercing the scalp, for example, as will be described in greater detail in the section “FEEDBACK/MONITORING”.

In an exemplary embodiment of the invention, needles 102 and/or discs 104 are arranged to allow existing hair on the scalp to be displaced (e.g., brushed) away from the needles during use, for example, as illustrated in FIG. 1B. Optionally, discs 104 are arranged parallel to one another, to allow hair to be brushed between the discs. Discs 104 are located about 1 mm apart, 3 mm apart, about 5 mm apart, or other smaller, intermediate or larger distances are used. A potential advantage is to move hair out of the way of needles 102, such that needles 102 can pierce the skin of the scalp.

In an exemplary embodiment of the invention, needles 102 are coated by at least one metal. Alternatively, needles 102 are made from the metal. Optionally or additionally, discs 104 are coated and/or made from the metal.

In an exemplary embodiment of the invention, the ionic form of the metal is a type I 5-alpha-reductase inhibitor, for example, copper 106 and/or zinc 108. Optionally, alternating discs 104 of copper 106 and zinc 108 are located in parallel. A potential advantage is release of the 5-alpha-reductase inhibitor, for example ions 158, directly into the skin of the scalp during piercing by needles 150 152, for example, as will be described in more detail in the section “5-ALPHA-REDUCTASE INHIBITOR”.

In an exemplary embodiment of the invention, needles 102 are coupled to a housing 110, for example, discs 104 are connected to an axle 112 to provide for rotation of discs 104. Optionally, housing 110 provides housing for discs 104, electrical contacts, heating elements and/or vibration elements.

In an exemplary embodiment of the invention, housing 110 is connected to handle 114. A potential advantage is that device 100 can be manually controlled by the user, for example by being hand-held.

In an exemplary embodiment of the invention, device 100 is light-weight, for example, less than 100 grams, less than 150 grams, less than 250 grams, or other smaller, intermediate or larger weights are used.

In some embodiments of the invention, device 100 is connectable to a base 116. Optionally, base 116 provides a power supply, for example, an alternating voltage and/or current such as from a wall outlet, and/or a direct voltage and/or current for example from a battery (e.g., rechargeable battery). Alternatively or additionally, base 116 provides a communication link to and/or from device 100, for example software to control device 100, and/or data gathered by device 100. Alternatively or additionally, base 116 may include an electronic controller. In some embodiments, at least some functions of base 116 are located in handle 114. For example, handle 114 may include electronics.

In an exemplary embodiment of the invention, device 100 is electrically connected to a user interface 118, for example, LED and/or a screen, for example to provide feedback to the user. Optionally, LEDs with different colors indicate status of device 100, non-limiting examples include; indicating when device 100 is connected to the power supply, indicating when the heating element of device 100 has reached the desired temperature and ready for use, indicating when device 100 is not yet ready for use (e.g., heating element is being heated). Optionally, a screen indicates status of device 100, non-limiting examples include; displaying the current usage (e.g., number of rotations on scalp), displaying the percentage of the treatment and/or dose delivered, warnings, error messages.

FIG. 1B is an illustration of the use of the device, for example device 100 of FIG. 1A, in accordance with an exemplary embodiment of the invention, for example, during the piercing of the scalp. FIG. 1B illustrates needles 150 and 152 (FIG. 1B) (e.g. part of array 102 (FIG. 1A)) piercing scalp 154 (FIG. 1B).

FIG. 1C illustrates scalp 154 after needles 150 and 152 have pierced scalp 154 and have been removed from scalp 154. In an exemplary embodiment of the invention, device comprises an array of needles 102. Needles 150 and 152 pierce scalp 154 forming wounds 156 surrounded by ionized tissue 166. More details about needles are provided herein, for example, in the section “MECHANICAL STIMULATION”.

FIG. 1D is a ‘face-on’ view of device 100 of FIG. 1A, in accordance with an exemplary embodiment of the invention.

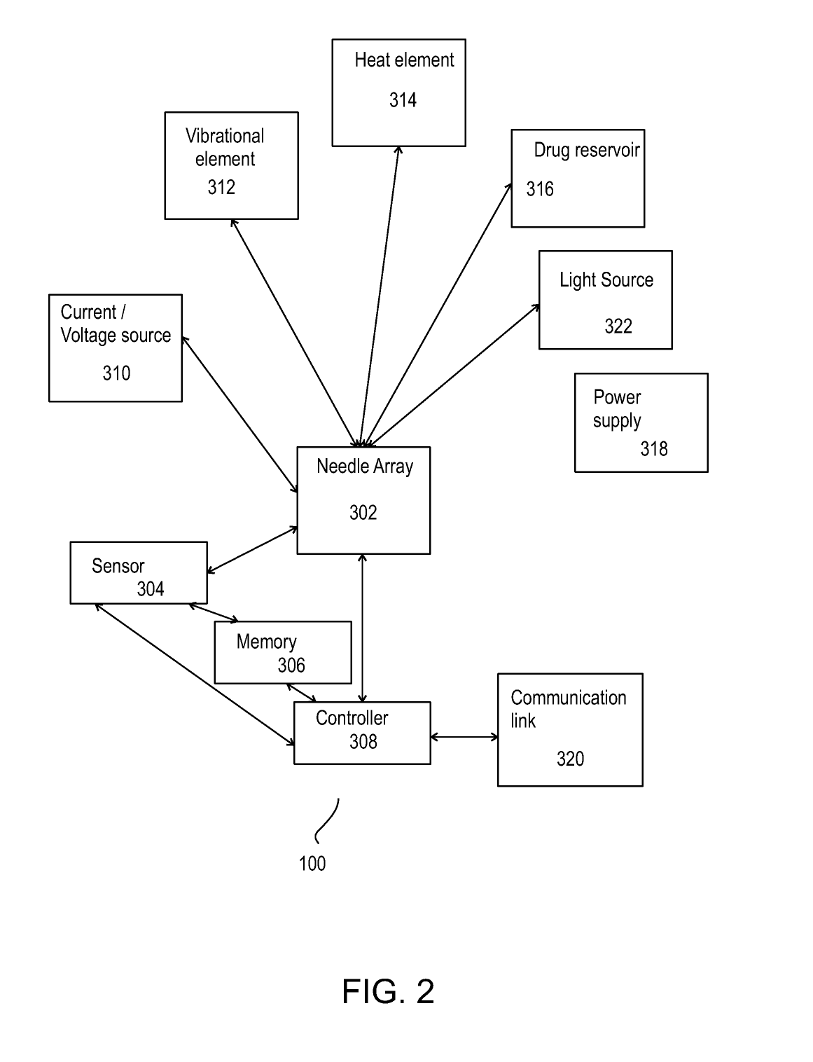

FIG. 2 is a block diagram of device 100 of FIG. 1, in accordance with some embodiments of the invention. Additional optional elements are illustrated.

In an exemplary embodiment of the invention, needles 302 (e.g., needles 102 (FIG. 1A)) are coupled to a current and/or voltage source 310, for example located in housing 110 (FIG. 1A). Optionally, springy rods, serving as electrical contacts, contact metallic rings on the axis. Optionally, each ring touches a disk to transmit current. For example, needle 150 is coupled to a first voltage V1 160 (FIG. 1B), and needle 152 is coupled to a second voltage V2 162, forming a voltage gradient in scalp 154. More details will be described, for example, in the section “ELECTRICAL STIMULATION”.

In some embodiments of the invention, needles 302 are coupled to at least one vibrational element 312, for example located in housing 110 (FIG. 1A). For example, needles 150 and 152 (FIG. 1B) are vibrated in one or more directions as illustrated by arrows 164 (FIG. 1B). Vibrations can result in increasing the volume of wound 156, shown by the dotted area. More details will be described for example, in the section “VIBRATION”. In some embodiments of the invention, a small DC motor 122 (shown in FIG. 1D) with a mass attached to its axis but a bit off-center vibrates needles 120 (FIG. 1D), for example, providing 2-dimensional vibrations (e.g., omni-directional) perpendicular to the axis of motor 122.

In some embodiments of the invention, needles 102 (FIG. 1A) are coupled to at least one heating element 314 (FIG. 2), for example located in housing 110 (FIG. 1A). For example, needles 150 and 152 (FIG. 1B) heat a volume of tissue 168 in scalp 154. More details will be described in the section “THERMAL STIMULATION”.

In some embodiments of the invention, device 100 (FIG. 1A) comprises a drug reservoir 316 (FIG. 2), for example located in housing 110 (FIG. 1A), for example, as will be described with more detail in the section “ADJUVANT TREATMENT”.

In some embodiments of the invention, device 100 (FIG. 1A) comprises a light source 322 (FIG. 2), for example, as will be described with more detail in the section “LIGHT STIMULATION”.

In some embodiments of the invention, device 100 comprises at least one sensor 304, for example located in housing 110. Optionally, sensor 304 detects the position of needle array 302 relative to the scalp, for example, using x and/or y coordinates across the surface of the scalp. Alternatively or additionally, sensor 304 is a temperature sensor to measure the temperature of the scalp. Alternatively or additionally, sensor 304 is an optical sensor to detect changes in skin color. Potentially, measurements of the temperature and/or skin color are used to estimate the inflammatory response and/or increase in blood flow, for example, as a result of treatment.

In some embodiments of the invention, device 100 comprises a controller 308, optionally located in housing 110, for example, as will be described with more detail in the section “CONTROLLER”.

In some embodiments of the invention, device 100 comprises a memory 306, optionally located in housing 110 and/or accessed by controller 308 and/or sensor 304, for example, as will be described with more detail in the section “CONTROLLER”.

In some embodiments of the invention, device 100 comprises a communication link 320, for example, to link controller 308 and/or memory 306 with a remotely located processor, non-limiting examples include; a computer, a laptop, a central server for example viewed by operators, a central database (e.g., access through the internet). Optionally, link 320 is wireless, non-limiting examples include; 802.11, blue tooth, wireless cellular phone network. Alternatively or additionally, link 320 is wired.

In some embodiments of the invention, link 320 is used to perform one or more functions, non-limiting examples include; upgrade controller 308 software, download usage data from memory 306, program controller 308 with the prescribed treatment.

In some embodiments of the invention, device 100 comprises a power supply 318. Optionally, power supply 318 is a plug. Alternatively or additionally, power supply 318 comprises one or more batteries, optionally rechargeable. Power supply 318 provides electrical power to one or more of; communication link 320, controller 308, memory 306, sensor 304, current/voltage source 310, vibration element, heat element 314, drug reservoir 316, needle array 302.

In some embodiments of the invention, elements 310, 312, 314, 316 are coupled to array 302, for example to apply a respective stimulatory modality through array 302, for example, underneath the skin surface. Device 100 can be comprised of any combination of elements 310, 312, 314, 316, for example none of the elements, any single element, any two elements, any three elements, or all of the elements.

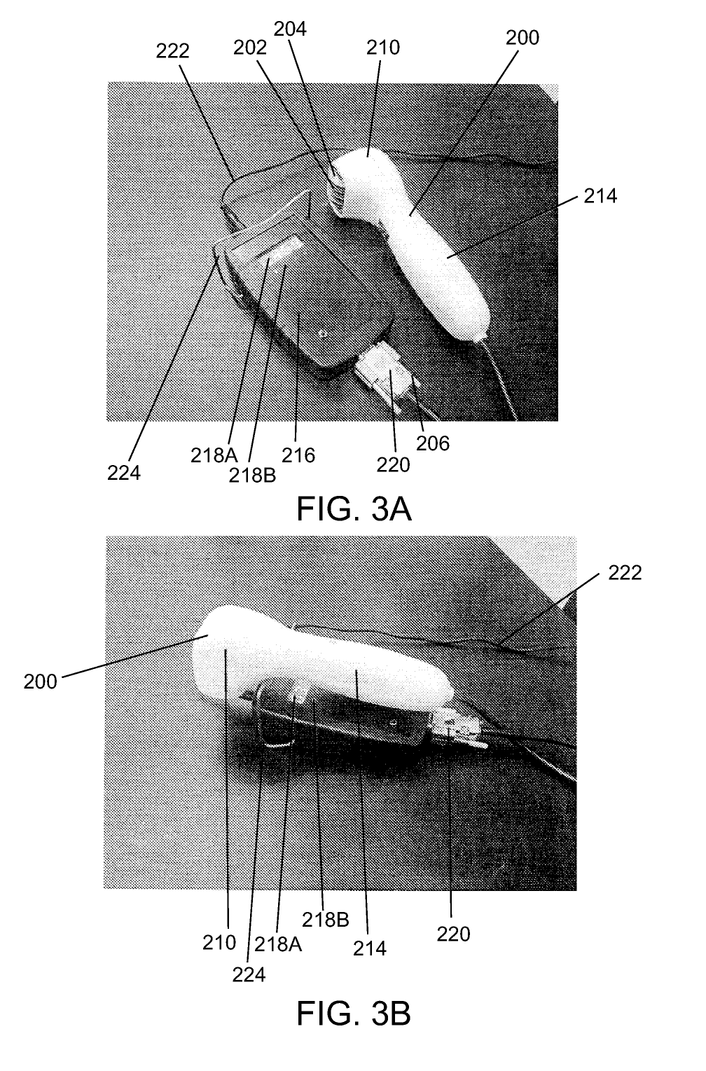

FIGS. 3A and 3B are images of a prototype device 200 to stimulate hair growth, useful for practicing some embodiments of the invention, for example, device 100 of FIG. 1. FIG. 3A is an isometric view of device 200, illustrating for example 8 alternating zinc and copper discs 204, each having needles 202 on the circumference. Optionally, discs 204 are located in housing 210. Optionally, device 200 is held by a handle 214. An optional on/off switch 206 is used to turn device 200 on or off. An optional cable 220 provides a communication link to base 216. An optional numerical display 218A is used to display a variety of data, for example number of rotations of discs 204. Optional color coded LEDs 218B display state data of the device, for example power, and/or heating of heating element. Optional cable 222 provides power to base 216 and/or device 200. An optional elevated wire 224 attached to base 216 provides for a rest position of device 200, potentially protecting needles 202 from contact damage (as shown in FIG. 3B).

FIG. 3C is an illustration of an exemplary disc, for example disc 204 as used in device 200 of FIGS. 3A-3B, useful for practicing some embodiments of the invention. Thickness of disc 204 (not including the needles) is, for example, about 0.1 mm or 0.07 mm, 0.05 mm, 0.03 mm, 0.13 mm, 0.15 mm, 0.2 mm, or other smaller, intermediate or larger values are used. Optionally, disc 204 thickness is uniform.

In an exemplary embodiment of the invention, one or more needles 252 are located around a circumference of disc 204. Spacing 246 between needles 252 as measured at the tips is, for example, about 1 mm, or 1.5 mm, 1.3 mm, 0.8 mm, 0.6 mm, or other smaller intermediate or larger values are used. Height of needles 248 is, for example, about 0.5 mm, or 0.7 mm, 1 mm, 0.3 mm, 0.1 mm, or other smaller, intermediate or larger values are used. Optionally, needles 252 have a substantially uniform cross section along a length, optionally tapering to a tip. Alternatively needles 252 taper to a tip along the length.

In an exemplary embodiment of the invention, needles 252 are fairly rigid, for example, to pierce the skin. Alternatively, needles 252 are fairly flexible, optionally omnidirectionally, for example, to relatively increase the cross sectional area of the wound beneath the skin.

In some embodiments of the invention, needles are flexible. Optionally, the entire needle is flexible. Alternatively, the portion of the needle piercing the skin is relatively inflexible, and the portion of the needle outside the skin is flexible. Potentially, flexibility slices the skin in unpredictable and/or random movements.

In an exemplary embodiment of the invention, an encoder (e.g. optical encoder) determines the amount of rotation of disc 204. Optionally, light from an LED passing through one or more distal apertures 244, located a distance 250 from the center of disc 204, is sensed by a sensor to determine the degree of rotation. Other types of rotational encoders can also be used, for example, an electrical encoder. Disc 204 has diameter indicated by a reference 240, for example, between 10 and 150 mm.

### Overview of Exemplary Method of Treatment

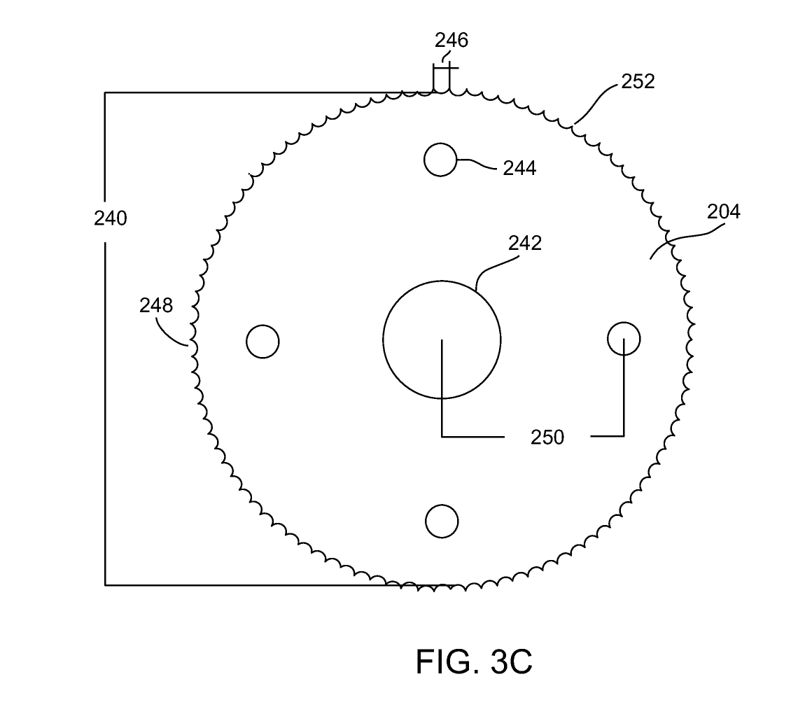

FIG. 4 is a flowchart of an exemplary method of stimulating scalp hair growth, in accordance with an exemplary embodiment of the invention. Optionally, the method uses the hair stimulation device, for example illustrated with reference to FIG. 1.

Optionally, at 402, a patient is selected, in accordance with an exemplary embodiment of the invention. Optionally, the patient is male. Optionally or additionally, the patient has been diagnosed with androgenic alopecia. Optionally, the patient is at the early stages of hair loss (e.g., has not lost most of his hair).

Optionally, at 404, the treatment plan is selected, in accordance with an exemplary embodiment of the invention. Optionally, a mechanical stimulation protocol is selected. Optionally or additionally, a vibration stimulation protocol is selected. Optionally or additionally, a thermal stimulation protocol is selected. Optionally or additionally, an ion deposition protocol is selected. Optionally or additionally, an electrical stimulation protocol is selected.

In some embodiments of the invention, at least some of the stimulation protocols (e.g., vibration, thermal, ion, electrical) are applied substantially simultaneously. Alternatively or additionally, at least some of the protocols are applied successively, for example, in no particular order. Alternatively or additionally, some protocols are selectively applied, while other protocols are not applied.

In some embodiments of the invention, the treatment plan is selected manually, for example by a physician, for example, based on personal experienced and/or clinical guidelines. Alternatively or additionally, the treatment plan is selected automatically, for example by software, for example, based on collected experimental data.

In some embodiments of the invention, the treatment plan is selected over a long period of time, for example, a single treatment session is to be repeated for a duration of time. For example, a single treatment plan is repeated four times a day, three times a day, twice a day, once a day, every other day, three days a week, twice a week, once a week, or other smaller, intermediate or larger time frames and/or repetition rates are used. For example, treatment is repeated over a month, over two months, over six months, over one year, over two years, indefinitely, or other smaller or intermediate time frames are used. Optionally, treatment is stopped when a desired growth effect is achieved and/or a certain time after, for example, a week or a month. Optionally or alternatively, stimulation is stopped, or at least paused for a week or more, if further progress is not seen. Optionally, the application and/or delay of treatment depends on scalp thickness, with treatment, for example, being continued as long as scalp thickness continues to increase and/or only if an increase is found.

In an exemplary embodiment of the invention, a maintenance level of treatment is defined and followed by the user.

In some embodiments of the invention, the treatment plan is selected so that a different part of the scalp is treated during different treatments. For example, treatment may be twice a day with a different part of the scalp treated during each of the two daily treatments. Optionally, the areas of treatment during different treatment sessions partially overlap.

In some embodiments of the invention, the time per session is selected. For example, about 30 seconds, 1 minute, 2, 4, 6, 10 minutes, or other smaller, intermediate or larger times or subranges thereof are used. Optionally, the time is selected according to a pain level caused by the device and/or a user pain and/or comfort threshold.

In some embodiments of the invention, the treatment area is selected. For example, approximately 50% of the total area in need of treatment, 10%, 25%, 33%, 67%, 75%, 90%, 100% or other smaller, intermediate or larger areas or subranges thereof are used.

At 406, the treatment plan and/or protocol is applied to the patient, in accordance with an exemplary embodiment of the invention. For example, the patient holds the device, and rolls the discs over the area of his scalp that requires stimulation. The needles on the discs prick his scalp according to the mechanical stimulation protocol. Optionally or additionally, the needles are vibrated according to the vibration protocol. Optionally or additionally, the skin is heated underneath the surface (e.g., heat transferred through the needles) according to the thermal stimulation protocol. Optionally or additionally, ions are deposited into below the skin (e.g., released from metallic coating on the needles) according to the ion deposition protocol. Optionally or additionally, electrical current and/or voltages are applied underneath the surface of the skin (e.g., using the needles as electrodes) according to the electrical stimulation protocol.

In a non-limiting example, a protocol comprises of treatments applied 3 times a week, for about 5 minutes per treatment. Each treatment comprises the following stimulations: 5 Volts, at 100 Hz AC, Zinc biased duty cycle, heating to a temperature of 60 degrees Celsius and vibration. Optionally, the protocol is selected according to trial and error, for example, the protocol is adjusted after a couple of weeks depending on the response of the scalp.

Optionally, at 408, the treatment is repeated, for example, according to the plan as in 404, in accordance with an exemplary embodiment of the invention. Optionally, the same treatment protocol is repeated. Alternatively, the treatment protocol is adjusted. For example, the initial treatment protocol is selected, the treatment is applied, and the treatment is adjusted based on feedback of success of the treatment.

### Exemplary Method of Treatment

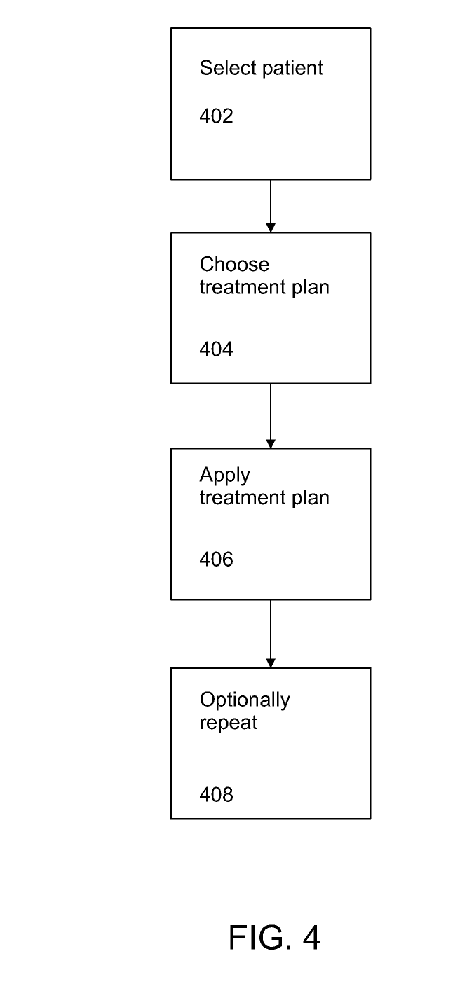

FIG. 5 is a detailed method of treatment of FIG. 4, in accordance with an exemplary embodiment of the invention.

Optionally, at 502, a patient is selected for treatment, for example, as will be described in the section “PATIENT SELECTION”.

Optionally, at 504, a decision is made with regards to the mechanical stimulation protocol, for example, as will be described in the section “MECHANICAL STIMULATION”.

Optionally, at 506 a decision is made with regards to the vibration protocol, for example, as will be described in the section “VIBRATION”.

Optionally, at 508 a decision is made with regards to the thermal stimulation protocol, for example, as will be described in the section “THERMAL STIMULATION”.

Optionally, at 510 a decision is made with regards to the ion application protocol, for example, as will be described in the section “ION APPLICATION”.

Optionally, at 512 a decision is made with regards to the electrical stimulation protocol, for example, as will be described in the section “ELECTRICAL STIMULATION”.

Optionally, at 522 a decision is made with regards to the use of adjuvant treatment, for example, as will be described in the section “ADJUVANT TREATMENT”.

Optionally, at 524 a decision is made with regards to the use of light stimulation, for example, as will be described in the section “LIGHT STIMULATION.”

Optionally, at 526 a decision is made with regards to the spatial and temporal parameters, for example, as described in the section “OVERVIEW OF EXEMPLARY METHOD OF TREATMENT” and as will be described in the section “MECHANICAL STIMULATION.”

Optionally, at least one of the parameters chosen in steps 504, 506, 508, 510, 512, 522 and 524 are specific per scalp area and are determined individually for each scalp area to be treated. For example, the temple area could receive one treatment and the vertex area could receive a different treatment. For example, it may be determined to treat the vertex area consecutively 5 minutes daily while the temples area is to be treated consecutively 4 minutes daily.

At 514, the treatment plan is applied, for example, as will be described in the section “APPLY TREATMENT”.

Optionally, at 516 feedback related to the treatment is obtained, for example, as will be described in the section “FEEDBACK”.

Optionally, at 518 one or more variables of one or more treatment protocols are adjusted, for example, as will be described in the section “ADJUSTING TREATMENT”. Optionally, the adjustment is related to the feedback as in 516.

Optionally, at 520 treatment is repeated, for example, as will be described in the section “REPEAT”.

### Potential Advantages

One or more embodiments have one or more potential advantages:
A plurality of stimulation modules can create a synergy to stimulate hair growth, for example, through one or more non-limiting mechanisms for example; triggering a wound healing response, inhibiting type I 5-alpha-reductase, electrical stimulation, increasing blood supply to the skin.
Treatment can be performed at home by the patient, for example when it is convenient for the patient. Treatment is easily repeatable.
Relatively low cost modality.
Relatively improved results, for example due to monitoring of the treatment for adherence and/or proper use. Feedback can be provided to the user, for example to guide the user in using the device properly.
Treatment can be selected to be painless, or to have a tolerable pain level. A potential advantage of pain-free or the tolerable pain level is that the patient is more likely to be compliant with use of the device.
Anesthesia is not required. For example, use of anesthetizing agents administered by a qualified practitioner is not required (e.g., general anesthesia, local injection of lidocaine).
Treatment can be selected to not result in bleeding and/or bruising. One or more potential advantages of the reduced inflammatory response and/or bleeding include; relatively increased comfort to the user, preventing a cosmetically unacceptable appearance (e.g., noticeable swelling and/or redness on the head), ability to repeat treatments within relatively shorter durations.

### Patient Selection

In an exemplary embodiment of the invention, patients are screened for treatment with the hair stimulation device and/or hair stimulation method.

In an exemplary embodiment of the invention, non-limiting examples of inclusion criteria include; males, diagnosis of androgenic alopecia, relatively early stages of hair loss. Use of the device and/or method is not limited to the described patients, as females can be treated and/or patients can be suffering from other disorders leading to hair loss can be treated.

In an exemplary embodiment of the invention, patients having factors that potentially interfere with treatment are excluded. Non-limiting examples include; genetic and/or inherited metallic ion metabolic disorders (e.g., Menkes disease, Wilson's disease [copper metabolism disorders]), electrically sensitive implanted equipment (e.g., brain stimulation device, pacemakers), use of medication (e.g., 5-alpha-reductase inhibitors for example finasteride for benign prostatic hypertrophy), history of tumors and/or cancer of the scalp and/or prostate.

In an exemplary embodiment of the invention, patients undergo an evaluation by a physician and/or other trained practitioner before treatment begins. Non-limiting examples of the evaluation include; a medical history (e.g., family history of hair loss), a physical exam (e.g., general signs of well being, signs of cancer on scalp, evaluation of amount of hair lost), laboratory tests (e.g., blood tests for copper and/or zinc levels), invasive tests (e.g., biopsy of scalp skin for evaluation).

In an exemplary embodiment of the invention, the physician selects one or more regions on the scalp for treatment. Optionally, all regions will be prescribed the same treatment protocol. Alternatively, different regions will be prescribed different protocols, e.g., if one region experienced more hair loss that another region.

### Mechanical Stimulation

In an exemplary embodiment of the invention, the scalp is mechanically stimulated, for example, a mechanical stimulation protocol is selected. The stimulation consists of selectively wounding the areas of skin where hair growth is desired (e.g., micro-trauma), such by one or more needles piercing the skin of the scalp.

Inventors hypothesize that selective wounding of skin on the top of the head (e.g., scalp) will lead to stimulation of hair growth, for example by initiating a wound healing response. Inventors hypothesize that hair will be stimulated through follicle regeneration (e.g., differentiation of stem cells in the skin of the scalp). Inventors hypothesize that sufficiently dense micro-trauma can have an effect of hair follicle regeneration following wound healing similar to that of contiguous trauma. The hypotheses are meant to be non-limiting, embodiments of the invention can still work even if the hypotheses are wrong.

In some embodiments, it may be effective to perform multiple localized treatments, for example, spaced at between 0.1 mm and 4 mm to overcome the over-localization of the skin mechanisms (for example, wound healing occurring in very small areas) and the over-sensitivity to the magnitude of the effect (for example, sensitivity to heat shock). Optionally, the use of multiple localized treatments some treatments, reduces treatment pain. For example, micro-wounding and/or micro-heating may produce less pain than wounding and heating a large area.

In an exemplary embodiment of the invention, the mechanical stimulation protocol comprises one or more variables. Non-limiting examples of selectable parameters include:
Depth of wound: In an exemplary embodiment of the invention, the wound is selected to be no deeper than the dermis layer of the skin of the scalp. Optionally, the wound extends past the epidermis into the dermis. Optionally, the wound is selected to have a depth of, for example, 50 micrometers to 1850 micrometers, or 70 micrometers to 1400 micrometers, or 100 micrometers to 1000 micrometers, or 400 micrometers to 600 micrometers, or other smaller, intermediate or larger ranges are used.
In some embodiments, the wounds are of multiple depths. For example, the wounds are created at depths of about 100 micrometers, about 300 micrometers and/or about 600 micrometers. Optionally, multiple depths are created by, for example, different length needles. Optionally or alternatively, some functions of the device are carried out at the epidermis and other functions are carried out at the dermis. For example, ions of opposing charge could be deposited not only in different locations, but in different depths. Optionally, the different depths enhance the electrical field effect. Alternatively or additionally, the depth of the wound is selected according to patient specific factors, non-limiting examples include; male or female, age of patient, degree of hair loss, for example, according to a table of correlation data values for example described by Hiroyuki et al.
Cross sectional area of individual wound: In an exemplary embodiment of the invention, the cross sectional area of an individual wound is selected to be, for example, about 1 mm², about 0.1 mm², about 0.01 mm², about 0.001 mm², about 0.0001 mm², or other smaller, intermediate or larger sizes are used. Alternatively or additionally, the volume of the individual wound is selected, for example, about 0.5 mm³, about 0.3 mm³, about 0.1 mm³, about 0.01 mm³, about 0.005 mm³, or other smaller, intermediate or larger volumes are selected.
Shape of individual wound: In an exemplary embodiment of the invention, the shape of an individual wound is selected, non-limiting examples include; cylinder, cone, pyramid, cross (‘+’).
Density of wounds: In an exemplary embodiment of the invention, the density of wounds per unit area of scalp to be treated is selected, for example, about 1 wounds/mm², about 5 wounds/mm², about 8, wounds/mm² about 10 wounds/mm², or other smaller, intermediate or larger densities are used.
Total area of wounding: In an exemplary embodiment of the invention, the area of scalp to be wounded from the total area of the scalp to be treated is selected. The area of wounding is selected to be, for example, about 1%, about 0.1%, about 0.01% of the area to be treated, or other smaller, intermediate or larger values are used.
Gaps between wounds: In an exemplary embodiment of the invention, the distance between wounds is selected. Optionally, the space between wounds along a first axis is selected. Optionally or additionally, the space between wounds along a second axis is selected, for example, the first and second axes are perpendicular to one another. In some embodiments, gaps along at least one axis are selected according to the existing amount of hair at the area to be treated, for example, relatively larger spaces are selected for a region with relative denser hair and/or hair having a relatively larger diameter. Existing hair may be displaced to the gaps between the wounds, so as not to interfere with skin wounding. Spaces between wounds along the first axis are selected to be about, for example, 3 mm, about 4.5 mm, about 6 mm, or other smaller, intermediate or larger spaces are used. Spaces between wounds along the second axis are selected to be, for example, about 0.3 mm, about 0.5 mm, about 1 mm, about 1.5 mm, about 2 mm, or other smaller, intermediate or larger values are used

In some embodiments of the invention, the distance between wounds and/or the density of wounds is selected according to the probability of hitting the follicles. Optionally, the density and/or distance between wounds is selected to reduce the probability of damaging follicles. Potentially, the use of optional flexible needles reduces the risk of damage to follicles, for example allowing the skin and/or needle to yield away, escaping puncture.

In an exemplary embodiment of the invention, the wounding profile is selected to have a pain level that can be tolerated by the patient. Optionally, the patient does not experience pain, for example, by sufficiently thin and/or short needles that reduce and/or prevent activation of pain relaying nerves at the skin. Alternatively, the patient experiences minor discomfort.

In an exemplary embodiment of the invention, the wounding profile is selected to initiate a relative minor inflammatory response, for example, relatively minor local edema, erythema and/or swelling that heal immediately, that heal, for example, in less than 5 hours, in less than 12 hours, in less than 1 day, in less than 2 days, in less than 3 days, or other smaller, intermediate or larger time periods are selected.

In an exemplary embodiment of the invention, the wounding profile is selected to not cause bruising (e.g., below skin) and/or bleeding (e.g., above skin). Alternatively, relatively minor bruising and/or bleeding is experienced.

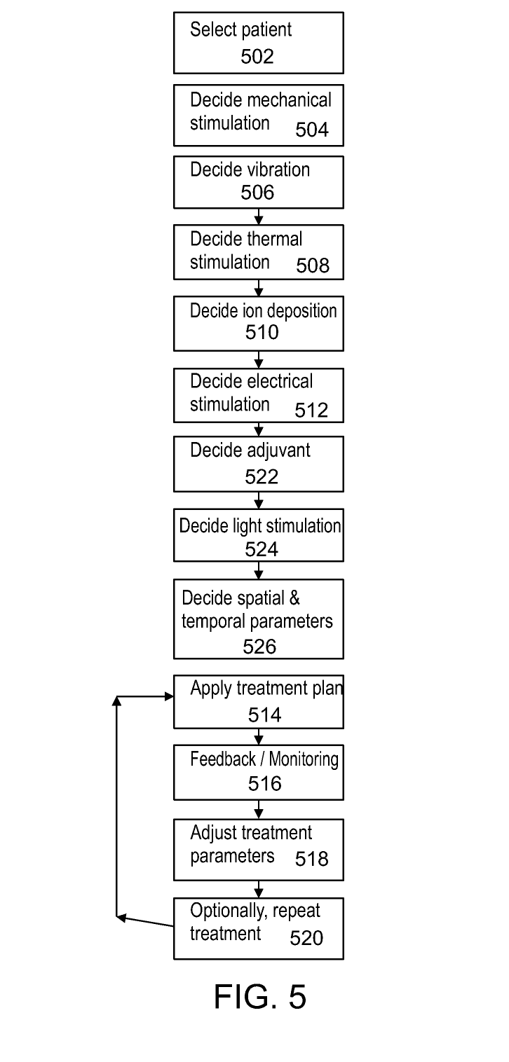

FIG. 6A is an illustration of an exemplary needle 600 used to cause microwounds in the scalp, for example, using embodiments of the needle array of the hair stimulation device as described herein.

FIG. 6B is a side view of a needle array 604 using needles 600 to cause a pattern of microwounds in the scalp 606.

FIG. 6C is a side view of a needle array 602 comprising needles 600 arranged along the circumference of discs 608 (e.g., replaceable and/or disposable).

Referring now to FIG. 6A, in an exemplary embodiment of the invention, needles, for example needles 600, are selected and/or arranged as an array according to the selected mechanical stimulation protocol. Non-limiting examples include; a cross sectional diameter 610 corresponding to the selected area of individual wounds, a length 612 corresponding to the selected depth of wound (optionally, a stopper 632, for example a flat disc, is used to set the needle length to prevent the needle from deeper penetration into the skin). Optionally or additionally, some embodiments comprise an array of needles comprising stoppers, each moving up and/or down independently, are used. Optionally or alternatively, some embodiments comprise at least two groups of arrays of needles comprising stoppers, each group moving up and/or down independently. In an exemplary embodiment of the invention, stopper movement is provided by a separate actuator (not shown), for example using a wheel which is parallel to the needle wheel and whose diameter is selectively changed and/or which is moved towards the skin using an eccentric.

In an exemplary embodiment, the actuator moves the needle up and/or down. Optionally, the actuator applies a force to the needle. Optionally, the stopper resists penetration with a force greater than the force of the actuator. Optionally, the resistance of the stopper limits the penetration of the needle. In an exemplary embodiment of the invention, the housing is designed to lean against the scalp. Optionally or alternatively, the wheel itself severs as a stopper to prevent over insertion of a needle.

In some embodiments, a group of needles is attached to a single actuator. Optionally, the combined force resisting penetration is higher than the force of the actuator. In some embodiments, at least one needle comprises a spring. Optionally, the spring helps prevent forceful penetration beyond the stopper's resistance. For example, the needle comprises an electronic spring probe (for example, Spring Contact Probes marketed by Allied Electronics, Inc.). Optionally, the probe structure with a spring acts as a safety mechanism and/or as a method to adhere to non-flat surfaces.

Optionally or additionally, needles are manipulated to result in the selected parameters of the mechanical stimulation protocol, for example, needles are vibrated (e.g., as described in the section “VIBRATION”) to relatively increase the depth and/or cross sectional area of the trauma, needles are repeatedly displaced over the scalp (e.g., rolled back and forth) for example to achieve the desired wound density.

In an exemplary embodiment of the invention, needles 600 have a round cross section (e.g., when taken perpendicular to the long axis). Alternatively, needles 600 can have other cross sectional shapes, for example square, oval, rectangular, square. Optionally, needles 600 have a uniform cross section, tapering to a tip. Alternatively, needles 600 are non-uniform, for example, tapering along the length.

In an exemplary embodiment of the invention, needles 600 of needle array 602 and/or 604 are selected with dimensions corresponding to one or more selected treatment protocol parameters. Non-limiting examples include; needles 600 a space 614 apart along discs 608 and/or discs a space 616 apart, and/or needles 600 a space apart 618 (FIG. 6C) along a first and/or second axis relative to other needles 600, corresponding to the selected gaps between wounds.

In an exemplary embodiment of the invention, a distance 626 and/or 628 between scalp 606 and device head 620 and/or 622 is set to provide a volume for hair 624 during penetration of needles 600 into scalp 606. Hair 624 can be displaced into the volume to let needles 600 pierce scalp 606 to allow the full length of needles 600 to enter. Distance 628 can be set for example, by diameter of discs 608 and/or by selecting the central hinge position within device head 620. Distance 626 can be set for example, by a selection of the total length of needle 600 (e.g., length 612 that forms wound and a region 630 (FIG. 6A) that does not pierce skin of scalp 606 (FIG. 6B)).

In an exemplary embodiment of the invention, the pattern of wounding is parallel straight lines, for example, for a roll of discs 608. Optionally, complex and/or random patterns of wounds can be created by repeated rolling of discs 608 over the scalp. Optionally, one or more discs each comprise multiple needles, arranged, for example, in a circumferential arrangement and/or along the thickness of the wheel, on the surface contacting the skin.

In an exemplary embodiment of the invention, needles 600 are made out of a biocompatible material, non-limiting examples include; metals (e.g., steel, silver, gold), glass, plastic, ceramic.

In an exemplary embodiment of the invention, needles 600 are coated with a type I 5a-reductase inhibitor, for example the metals zinc and/or copper.

### Vibration

In an exemplary embodiment of the invention, the scalp is additionally mechanically stimulated by vibration, for example, a vibration protocol is selected. One or more needles for example needles 600 described with reference to FIG. 6A are vibrated, for example, by a vibration element (e.g., actuator) for example element 312 described with reference to FIG. 2. Needles are vibrated at least during piercing of the skin (e.g., when causing microwounds, for example according to the mechanical stimulation protocol).

Inventors hypothesize that vibration of needles during microwounding will lead to stimulation of hair growth, for example, by relatively increasing the wound healing response due to relatively increased wound volumes. Investors hypothesize that vibration can also cause increased blood flow at the wound, for example by massaging the area, leading to relatively increased hair growth due to the higher amount of nutrients available to the hair. The hypotheses are meant to be non-limiting, embodiments of the invention can still work even if the hypotheses are wrong.

In an exemplary embodiment of the invention, the vibration protocol comprises one or more variables. Non-limiting examples of selectable parameters include:
Axis of vibration: In an exemplary embodiment of the invention, needles 600 are vibrated along the long axis of the needle. Alternatively or additionally, needles 600 are vibrated along at least one axis substantially perpendicular to the long axis (e.g., single axis parallel to direction of rolling disc 608, single axis perpendicular to direction of rolling disc 608, omni-directional).
Frequency of vibration: In an exemplary embodiment of the invention, the frequency of vibration of needles 600 is, for example, about 50 Hz, about 70 Hz, about 100 Hz, about 120 Hz, or other smaller, intermediate or larger frequencies are used.
Amplitude of vibration: In an exemplary embodiment of the invention, the amplitude (e.g., peak to peak) of vibration is, for example, about 0.05 mm, about 0.1 mm, about 0.2 mm, or other smaller, intermediate or larger amplitudes are used. Alternatively, the amplitude of vibration is, for example, about 50% of the diameter of the needle, or about 100%, about 200%, or other smaller, intermediate or larger amplitudes are used.

In an exemplary embodiment of the invention, the amplitude of vibration of needles 600 along the long axis is used to control and/or adjust the depth of the wound, for example according to the mechanical stimulation protocol.

In an exemplary embodiment of the invention, the amplitude of vibration of needles 600 along one or more axes perpendicular to the long axis is used to control and/or adjust the volume and/or cross sectional area of individual wounds, for example, according to the mechanical stimulation protocol.

A potential advantage of controlling the vibration is that the selected depth of wound and/or area of individual wounds can be controlled and/or adjusted (e.g., dynamically) without having to replace the needle array.

### Needle Actuators

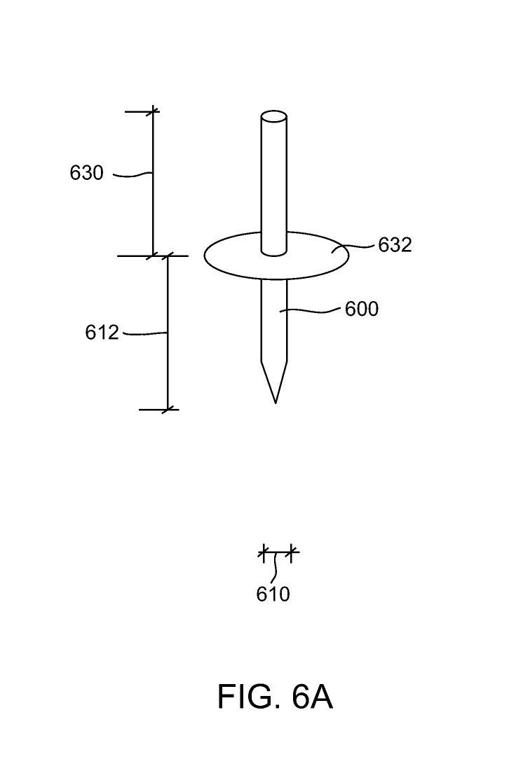

FIGS. 7A-7F are illustrations of embodiments of needle actuators, in accordance with some embodiments of the invention. Optionally, needle actuators act as vibrational elements, to vibrate needles according to the selected vibrational protocol.

In some embodiments of the inventions, one or more non-limiting examples of actuators include; piezoelectric elements, motorized linear actuators, and/or shape memory alloy actuators.

In some embodiments of the invention, needles are individually vibrated. Alternatively or additionally, groups of needles are vibrated together. Optionally, vibration is performed by an off-axis spinning mass, for example, the direction of the axis determines the plane of vibration. For example, translating the movement to a linear direction, pushing on a piston mass creates a linear vibration.

FIG. 7A is an isometric view, and FIG. 7B is a cross sectional view of a needle array 702, for example described with reference to FIG. 6C. Each needle 700 (e.g., as described with reference to FIG. 6A) of array 702 is coupled to an actuator 704. Optionally, each needle 700 is coupled to a separate actuator 704. Optionally, actuators 704 are attached to a power control 705.

FIG. 7C is an isometric view, and FIG. 7D is a cross sectional view of a needle array 706. Two or more needles are controlled by actuators, for example, array of nine needles 708 is controlled by actuator 710 and, for example, array of needles 706 is controlled by actuator 711. There are two or more groups of needles, for example, four groups 708, 730, 732 and 734 of nine needles in each group are controlled by four actuators 710, 736, 738 and 740.

Needle groups can be arranged in a variety of patterns. Non-limiting examples include the checkerboard pattern as illustrated in FIG. 7D, a bull's eye pattern as illustrated in FIG. 7E and/or a side by side tile pattern as illustrated in FIG. 7F. For example, the bull's eye pattern (FIG. 7E) may comprise one needle 715 in an inner circle and at least two needles in needle array 717 in an outer circle and, for example, the side by side tile pattern (FIG. 7F) may comprise eight groups 721, 722, 723, 724, 725, 726, 727 and 728 of needles.

In some embodiments, at least two groups (FIG. 7F) may touch the scalp simultaneously. For example, the device is configured so that several actuators receive a signal to “lower” and touch and/or penetrate the scalp simultaneously. Optionally or alternatively, several needles are connected to a single actuator 710 and go up and down together. Optionally, the needles conform (or are advanced to conform) to the scalp curvature and penetrate together. In some embodiments, the needles are equipped with a spring to facilitate conformity to the scalp curvature.

In an exemplary embodiment, 721 and 722 may touch the scalp simultaneously, 722 and 723 may touch the scalp simultaneously, or 723 and 724 may touch the scalp simultaneously, or 724 and 725 may touch the scalp simultaneously, or 725 and 726 may touch the scalp simultaneously, or 721, 722 and 728 may touch the scalp simultaneously, or 722, 725 and 727 may touch the scalp simultaneously or another combination of groups may touch the scalp simultaneously. Optionally, more than two types of ions are discharged from the needles. FIG. 7G is an isometric view of a single injector. Optionally, the injector comprises needle 700. Optionally, needle 700 is coupled to actuator 704. Optionally, actuator 704 comprises a substance to be injected. Optionally, the substance to be injected is ionized Zn solution. Alternatively, the substance to be injected is ionized Cu solution. Alternatively, the substance to be injected is a different ionized solution. Optionally, the injector provides heat. Optionally, the injector provides vibration

FIG. 7H is an isometric view of a 1-dimensional array of injectors. Optionally, at least two types of ion solution are contained in the injectors. For example, injector 750 comprises Zn solution. For example, injector 752 comprises Cu solution.

FIG. 7I is an isometric view of a 2-dimensional array of injectors. For example, the array may comprise three rows of four injectors each. Optionally, at least two types of ion solution are contained in the injectors. For example, injectors with actuators shaded with one grey density comprise Zn solution. For example, injectors with a different shown grey density actuators comprise Zn solution.

In some embodiments of the invention, the needle actuation pattern is selected to relatively increase stimulation in certain areas, for example ion injection. Optionally, the pattern is selected according to the follicular density of areas of the scalp. Alternatively or additionally, the pattern is selected to perform different stimulations at different areas simultaneously, with relatively reduced interaction.

In some embodiments of the invention, the entire length of the needle enters the actuator, for example during rest (e.g., none vibration mode) and/or during a part of the vibrational phase. A potential advantage is that the device head can be placed against the scalp without piercing the skin.

Potential advantages of vibrating the needle include one or more of:
Parting hair on the scalp to allow the needle to reach the skin.
Increasing the life of the needles, for example reducing wear and tear. For example, by changing the axis of vibration every set period of time, different areas of the needle experience wear and tear. Potentially reducing the increased wear caused by vibration.
Improving electrical contact below the skin surface, for example to relatively improve the application of an electrical current and/or heat transfer.
Improving mechanical contact and/or electrical contact underneath the skin surface, for example to improve ion injection into the skin (e.g., higher dispersion).
Increasing the microtrauma mechanical stimulation.
Reducing pain, for example by reducing the amount of time the needle pierces the skin.

### Thermal Stimulation

In an exemplary embodiment of the invention, the scalp is thermally stimulated, for example, a thermal stimulation protocol is selected. The thermal stimulation consists of selectively heating the areas below the surface of the skin where hair growth is desired, such by one or more needles piercing the skin of the scalp, for example, as described herein. The thermal stimulation is selected to cause micro-trauma to the skin, for example, by denaturing proteins.

Inventors hypothesize that selective micro-trauma to the skin by heating would induce a positive effect on the scalp skin, for example, thickening the skin, and/or promoting regeneration of degenerated hair follicles and/or new hair follicles. The hypotheses are meant to be non-limiting, embodiments of the invention can still work even if the hypotheses are wrong.

In an exemplary embodiment of the invention, the thermal stimulation protocol comprises one or more variables. Non-limiting examples of selectable parameters include:
Target temperature of skin: The desired temperature of the skin surrounding the wound, after the area has been heated by the needle. In an exemplary embodiment of the invention, the desired temperature is selected to be, for example, about 40 degrees Celsius, about 50, about 55, about 60, about 70 degrees Celsius, or other smaller, intermediate or larger temperatures are used.
Volume of heated skin: The dimensions of the resulting micro-trauma due to heating, for example by reaching the target temperature and/or reaching a coagulation temperature (e.g., above 55 degree Celsius). In an exemplary embodiment of the invention, the volume of traumatized skin is selected to be confined to the dermis (e.g., not to extend to the epidermis and/or subcutaneous layers). In an exemplary embodiment of the invention, the thickness of the volume of traumatized skin (across the dermal layer) formed around the needle (e.g., cylinder of skin around needle) is selected, for example, to have a thickness of about 0.01 mm, about 0.05 mm, about 0.1 mm, about 0.2 mm, or other smaller, intermediate or larger thicknesses are used. In some embodiments, the total volume of the skin to be traumatized is selected, for example, to have a volume of about 0.01 mm³, about 0.03 mm³, about 0.05 mm³, or other smaller, intermediate or larger values are used.
Pattern of heated skin: In some embodiments, not all needles heat the skin. For example, skin may be heated by alternating needles. Alternatively or additionally, different needles heat the skin different amounts.

In an exemplary embodiment of the invention, the volume of skin heated to the target temperature is associated with one or more needle parameters and/or other stimulation protocols, for example, the depth of the needle into the skin, the diameter of the needle, the spaces between needles, the vibrational frequency of the needles (e.g., the amount of time the needle is in contact with the skin) and/or the pattern of needles heating the skin. In some embodiments, the needles and/or other factors are selected according to the selected thermal stimulation protocol. Alternatively, the application of heat to the skin to achieve the desired thermal stimulation protocol is planned according to the selected needle and/or other stimulation protocols.

In an exemplary embodiment of the invention, the volume, pattern and/or target temperature are selected so as to enhance ion and/or medicament transport in desired locations.

In an exemplary embodiment of the invention, needles are thermally coupled to a heat source, for example element 314 as described in FIG. 2. One or more non-limiting examples of heat sources include; resistors (for example heating wires), lamps, light emitting diode (LED), laser. Optionally, the needles are heated inside a chamber for example a cradle until the needle reaches the desired temperature. Alternatively or additionally, the needle is directly heated, for example by internal resistance of the needle itself.

In an exemplary embodiment of the invention, the heat source maintains the temperature of the needle at the desired temperature, for example during pricking of the skin and/or during rest periods.

In an exemplary embodiment of the invention, heat sources are located internally (for example to housing 110), for example, integrated with and/or as a separate part of axle 112 and/or the needles actuators. Alternatively or additionally, heat sources are located externally (for example to housing 110), for example at the lower surface of the housing relatively close to the needle and/or skin surface.

In some embodiments of the invention, heat sources are not thermally coupled to the needles, for example, the heat source (e.g., laser) is used to cause an area of thermal damage independent of the needle piercing the skin.

In an exemplary embodiment of the invention, the heat capacity of the heat source is relatively large compared to the heat capacity of the volume of skin to be heated and/or to the heat capacity of the needles. Optionally, the heat source transfers heat substantially continuously to the needles, for example, during the pricking of the skin by the needles. Optionally, the heat source transfers enough heat to the needles to overcome heat loss for example to the skin, the air and/or the hair (by e.g., radiation and/or conduction).

In an exemplary embodiment of the invention, the needles are made out of a heat conductive material, for example metal.

In an exemplary embodiment of the invention, the heating of the skin by the needles begins to feel uncomfortable if the user stops rolling and/or pricking the scalp. Optionally, the heat transferred to the skin is not sufficient to cause burns beyond the selected heating profile. Removing the device from the skin stops the heat transfer.

A potential advantage of the heat source is that the temperature of the volume of skin to be wounded can be rapidly elevated to the target temperature. Another potential advantage is that the temperature of the volume of skin to be traumatized is maintained at the target temperature for a sufficient length of time to induce the desired wound to the desired volume of skin. A potential advantage of relatively quickly heating relatively small volumes of tissue to the target temperature is preventing and/or reducing pain and/or burns.

### 5-Alpha-Reductase Inhibitor Deposition

In an exemplary embodiment of the invention, the scalp is stimulated by depositing one or more materials into the skin, for example, an ionic deposition protocol is selected.

In some embodiments, subcutaneous treatment increases ion penetration by accessing ionic channels running through sweat ducts (e.g., Grimnes, Pathways of Ionic Flow through Human Skin in vivo), for example, by “hitting” the sweat ducts with the needles, without puncturing the stratum-corneum (SC). At a density estimated at 200˜400 glands/cm̂2 of skin on the scalp and a diameter of up to 50 micrometers, sweat ducts comprise less than 0.5% of the scalp surface. In some embodiments, the probability of a needle creating ionic current is increased by increasing the contact area of the needles with the scalp, for example, by increasing the cross-section and/or density of the needles. In some embodiments, the probability of a needle creating ionic current is increased by configuring the needles to physically penetrate the sweat ducts through the SC.

In an exemplary embodiment of the invention, the deposited materials have the property of inhibiting the enzyme 5-alpha-reductase. Optionally, type I 5-alpha-reductase is inhibited, for example, selectively inhibited to a greater extent than type II 5-alpha-reductase.

Without being bound to theory, 5-alpha-reductase is the enzyme which converts testosterone into the potent form dihydrotestosterone. Type I 5-alpha-reductase is expressed mainly in the skin, whereas type II 5-alpha-reductase is expressed mainly in the prostate. Dihydrotestosterone is believed to inhibit hair growth on the scalp.

Therefore, inventors hypothesize that selectively depositing ions that inhibit type I 5-alpha-reductase into the skin of the scalp will promote hair growth. However, the efficacy of some embodiments of the invention can be unrelated to the underlying theory, and work even if the theory is incorrect.

In an exemplary embodiment of the invention, the vibration protocol comprises one or more variables. Non-limiting examples of selectable parameters include:
Type of Material: The type I 5-alpha-reductase inhibitor is selected. In an exemplary embodiment of the invention, the type I 5-alpha-reductase inhibitor is one or more types of metals. Optionally, the metal is in ionic form. Optionally, the ions are relatively potent inhibitors. Optionally, the ions are relatively safe for deposition into the skin. Non-limiting examples of ions include copper, zinc, cadmium, nickel and iron. In an exemplary embodiment of the invention, the ions are relatively non-toxic and/or have a relatively higher affinity to type I 5-alpha-reductase, for example, copper and/or zinc.
   In some embodiments, ions create electrical fields. Optionally, the electric field is created between ions of different electrical potential and/or charge. Optionally, the electric field causes a change in ionic charges in and/or near the skin. Optionally or alternatively, the scalp is enriched with mineral nutrients. For example, the, ions enrich the scalp with micro-elements essential for health. For example, Zn, Cu and/or other elements may contribute to the health of the skin of the scalp. Optionally, avoiding a deficiency in Zn, Cu and/or other elements essential to the health of the scalp may avoid and/or reverse hair growth disorders.
   In some embodiments, deficiencies in Zn, Cu and/or other elements essential to the health of the scalp are detected in blood tests. Optionally, additives, for example, ions, are topically administrated, for example by ionic deposition, directly to the scalp. Optionally, additives are administrated to the entire the entire scalp systematically. Optionally, different areas of the scalp are evaluated for their need for health-essential micro-elements. Optionally, additives are topically administrated directly to the area of the scalp requiring them.
Dose: In an exemplary embodiment of the invention, the dosing schedule is selected. The dosing schedule comprises of the concentration of ions and/or number of ions to deposit in the scalp per unit of time (e.g., per needle penetration point, per area of scalp requiring treatment). For example, the dose of zinc is selected to be about 0.001, about 0.01, about 0.1, about 1 nanograms/cm² per treatment, or other smaller, intermediate or larger values are used. For example, the dose of copper is selected relative to the dose of zinc, for example, about 1%, about 10%, about 50% of the zinc dose. For example, the dose of copper is selected to be about 0.0001, about 0.001, about 0.01, about 0.1 nanograms/cm² per treatment, or other smaller, intermediate or larger values are used. In an exemplary embodiment of the invention, the number of ions delivered per needle can be controlled by controlling the charge that passes through the needle during the time that the needle pierced the skin, for example, by regulating the time the electrical pulse is turned on and off and/or by controlling the time that the needle is inside the skin.
   In an exemplary embodiment of the invention, the cumulative dose (for one or more of the ions) per day is 0.01 nanograms/cm². Optionally or alternatively, the maximum daily dosage is 0.5 nanograms/cm². Optionally or alternatively, the cumulative dose per week is 0.02 nanograms/cm². Optionally or alternatively, the maximum weekly dosage is 3 nanograms/cm².
   In an exemplary embodiment of the invention, the type of ions delivered per needle can be controlled by controlling the voltage of each coated needle.
   In an exemplary embodiment of the invention, the ratio between two or more materials (e.g., zinc:copper) injected beneath the skin surface is selected. For example, about 20:1, about 10:1, about 5:1, about 1:1, or other smaller, intermediate or larger ratios are used.
   In an exemplary embodiment of the invention, the ionic charge transferred beneath the skin surface is selected (e.g., total charge over the treatment). For example, the total transferred charge is about 5000, about 10 000, about 50 000, about 100 000, about 250 000 nano-Coulombs, or other smaller, intermediate or larger values are used. Non-limiting factors (one or more are selectable) affecting the total charge include; duration of the treatment, the voltage, the type of ions, the number and/or type of needles, the quality of contact between the skin and the needles, needle penetration depth.

In some embodiments, the number of ions deposited during treatment is controlled by adapting the voltage (see, for example, the methods described in Chizmadzhev et al, Electrical Properties of Skin at Moderate Voltages: Contribution of Appendageal Macropores), by adapting the temperature (see, for example, the methods described in Maulsby et al, The interrelationship between the galvanic skin response, basal resistance, and temperature), and/or by adapting the frequency. Increasing the voltage, temperature and frequency can each increase the number of ions deposited. For example, the number of ions deposited during treatment is controlled in an open loop manner by determining the voltage before beginning treatment. Alternatively, the number of ions deposited during treatment is controlled in a closed loop manner by determining the voltage during the treatment based on feedback received from sensors incorporated into the device.

In some embodiments, controlling the ions deposited is done directly by measuring the charge of each polarity (ion type), for example, by measuring and integrating the current passed through each type of disk set. The existence of current indicates the unit is in actual use. A degradation of current indicates a faulty unit, improper contact, or other means. Excessive current might indicate a faulty unit, or excessive moisture on the scalp (and therefore not enough current through the scalp).

In some embodiments, the mass of metal ions discharged from the electrodes may be calculated by a formula. For example, assuming the charge C is ionic, and the oxidation state Z, the mass m of metal ions discharged from the electrodes (w is the atomic mass, e the electron's charge, Na is Avogadro's number) is computed as follows:

$$m = \frac{C \cdot w}{e \cdot Z \cdot {N}_{a}}$$

In some embodiments, ion injecting electrodes that touch the scalp are connected to one terminal of a power source and an electrode that does not touch the scalp is connected to a second terminal of the power source. For example, the electrode that does not touch the scalp may be connected to a part of the body other than the scalp. For example, the device may comprise a handle comprising an electrode designed to touch the palm of a person holding the handle.

In some embodiments, the efficiency of the deposition of ions is enhanced, for all users or for a specific user, by performing a “calibration phase” in which the same region is treated for a period of a time while changing each parameter slightly and measuring the real-time response in current. Optionally, different treatment parameters may be chosen for different scalp areas of same user. Optionally, different treatment parameters may be chosen for different users.

In some embodiments, the efficiency of the deposition of ions is enhanced through general improvements in the parameters, for example, preparing a better cross section of the needles and/or starting with more efficient voltage and frequency. Optionally, the efficiency of the deposition of ions is enhanced through dynamic modification of changeable treatment parameters through closed-loop feedback/control.

In some embodiments, ion penetration increases blood flow when the electrical fields generated by the small charge deposits create a MENS (microcurrent electrical neuromuscular stimulation) effect in the skin. Optionally, the MENS effect shortens skin healing times. Optionally, the electrical fields invigorate movement of essential ions and stimulate the skin systems into an increased rate of activity.

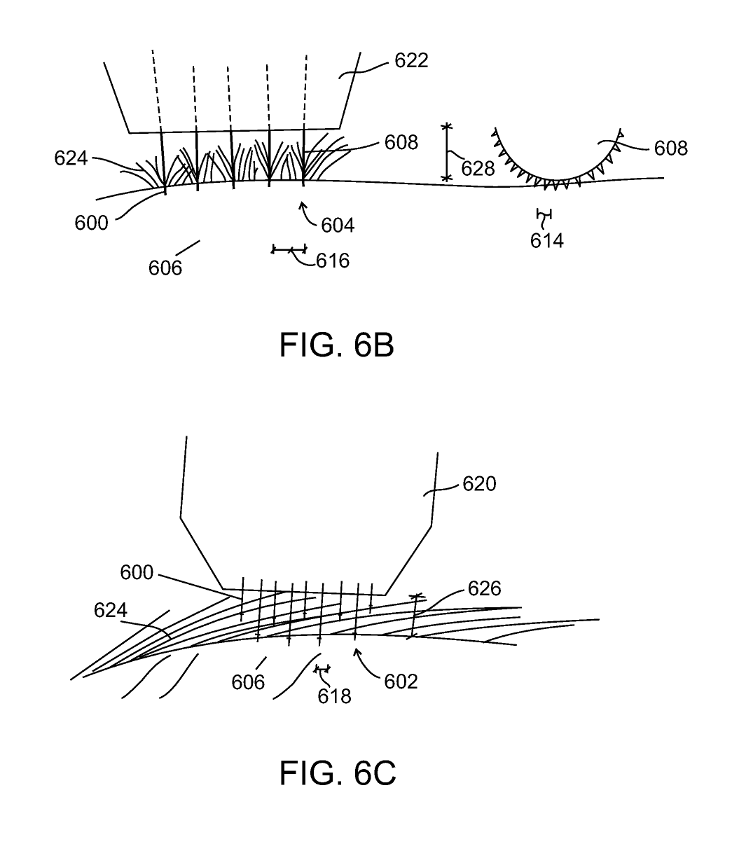

FIG. 8A is an illustration of an array of needles 802 depositing materials 804 beneath the skin 806 surface of scalp, in accordance with an exemplary embodiment of the invention. For simplicity purposes, array 802 comprises four needles 808, having the material 804 to deposit located at the part of the needle 808 that pierces scalp 806.

In an exemplary embodiment of the invention, needles 808 are made of material 804. Alternatively, needles 808 are coated with material 804. Optionally or alternatively, 830, 832, 834 and/or 836 represent electrical potentials which may exist on needles 808.

In an exemplary embodiment of the invention, two different needles 808 to be electrically coupled have two different materials 804 at their ends. For example, alternating discs (e.g., as illustrated in FIG. 1) are made from different materials, for example, copper and zinc.

In some embodiments, scalp 806 acts as a bridge, placing two needles having dissimilar metals in electrical contact. The metals can undergo galvanic corrosion, where one metal dissolves in scalp 806, while the other metal absorbs ions from scalp 806. For example, if one metal is zinc and the other metal is copper, the zinc will dissolve and the copper will accumulate. Optionally, material 804 is chosen to have other depositing effects. Optionally or additionally, current is forced in the opposite direction.

FIG. 8B is an illustration of ion deposition into scalp 806 for example using a galvanic cell set-up, in accordance with an exemplary embodiment of the invention. Optionally, a power source 812 (e.g., source 310 as described with reference to FIG. 2) electrically couples a first needle 814 and a second needle 816. For example, each needle 814 and 816 may have coated electrodes comprising different materials at the piercing ends 815 and 817, for example, needle 814 pierces scalp 806 at piercing end 815 with zinc and needle 816 at piercing end 817 with copper. Optionally, power source 812 emits Alternating Current (AC). Optionally, power source 812 emits Direct Current (DC).

FIG. 8C is an illustration of using the set-up as in FIG. 8B to release zinc ions into scalp 806, in accordance with an exemplary embodiment of the invention. The positive pole of power source 812 is electrically connected to needle 814 with zinc (e.g., acting as the anode 840), and the negative pole is electrically connected to needle 816 with copper (e.g., acting as the cathode 842). Zinc ions 819 are discharged from needle 814 into scalp 806, and copper ions 821 and/or other ions 823 are accumulated from scalp 806 onto needle 816.

In an exemplary embodiment of the invention, the voltage of power source 812 as in FIG. 8C is, for example, about 1V, about 3V, about 5V, about 7V, about 10V, or other smaller, intermediate or larger values are used.

FIG. 8D is an illustration of using the set-up of FIG. 8B to release copper ions into scalp 806, in accordance with an exemplary embodiment of the invention. The positive pole of power source 812 is electrically connected to needle 816 with copper (e.g., acting as the anode 850), and the negative pole is electrically connected to needle 814 with zinc (e.g., acting as the cathode 852). Copper ions 821 are discharged from needle 816 into scalp 806, and zinc ions 819 and/or other ions 823 are accumulated from scalp 806 onto needle 814.

In an exemplary embodiment of the invention, the voltage of power source 812 as in FIG. 8D is at least greater than the standard potential for the reaction, for example, above 1.10 Volt.

In an exemplary embodiment of the invention, power source 812 is an alternating current source. The frequency of source 812 can be selected to result in a desired ion deposition pattern, for example alternating between the set-ups as described in FIGS. 8C and 8D. For example, the frequency of source 812 is selected to be substantially half of the rate of needle pricks per second, for example when using the hair stimulation device with rolling discs, for example, as described with reference to FIG. 1. For example, if the device is rolled over the scalp to achieve a rate of skin pricks of 30 pricks per second and the frequency of source 812 is 15 Hz, the ions deposited during each needle prick will alternate, for example between copper and zinc. Furthermore, different ions will be deposited at different locations.

In some embodiments of the invention, the AC waveform (e.g., duty cycle) is selected according to the ratio of the desired material deposition. For example, to achieve a 10:1 ratio (e.g., of zinc:copper), a waveform having a 10:1 ratio (91% duty cycle) is selected. Alternatively or additionally, the number of needles coated with each material is selected according to the desired deposition ratio, for example, the number of needles coated with zinc relative to the number of needles coated with copper is 10:1.

In some embodiments of the invention, power source 812 is a direct current source. The polarity of source 812 can be selected to result in a desired ion type and/or deposition pattern. For example, according to the set-ups of FIGS. 8C and/or 8D. The set-up of FIG. 8C can also be achieved without source 812, for example by electrically connecting needle 814 and 816.

In an exemplary embodiment of the invention, ions are deposited below the scalp by jet injection carried out by at least one jet. Jet injection is a widely available technology (for example, Taberner et al, Needle free jet injection using real-time controlled linear Lorentz force actuators, The Medical Jet Injector marketed by AMI (Advanced Meditech International), Inc; Rhodes, Shallow Dermal Delivery of Vaccines Using Jet Injectors) and may be adapted for use with the instant application, in accordance with some embodiments of the invention.

In an exemplary embodiment of the invention, one jet injects Zn ions and a second jet injects Cu ions. Optionally, additional jet injectors are used to speed the process of ion deposition. Optionally or alternatively, additional or alternative jets are used to inject other types of ions. Optionally, each jet injector contains a different solution. Optionally, each jet injector deposits one type of ion solution at a specific location.

In some embodiments, materials are injected in controlled small distances between points of entry. Optionally, the injectors are configured on a flat plane, or on a rounded surface that rolls along the scalp.

In some embodiments, multiple-material injectors known in the art (for example, U.S. Pat. No. 8,048,019 to Nisato et al) inject multiple materials at the same location. Optionally, the material in the jet is heated, for example, to between 55 and 65 degrees Celsius or higher) to cause micro-burns.

In some embodiments, ions are deposited below the scalp by direct injection using a hollow needle containing ionized solution.

In some embodiments of the invention, materials (e.g., ions) are added directly to the scalp, for example in the form of a lotion, gel and/or water. Non-limiting examples of ions in this form include ZnSO₄, CuSO₄. The lotion can be added in addition to the use of coated needles, or instead of coated needles (e.g., using uncoated needles). Optionally, the ions penetrate below the surface of the skin due to piercing and/or vibration of the needles. Alternatively or additionally, the ions penetrate the skin due to an iontophoretic effect created by the electrical charges on the needles.

### Electrical Stimulation

In an exemplary embodiment of the invention, the scalp is stimulated by applying one or more currents and/or voltages to areas of the skin, for example, an electrical stimulation protocol is selected. Optionally, a plurality of currents and/or voltages are applied to the scalp, for example different voltages and/or currents to different areas and/or between different needles.

In an exemplary embodiment of the invention, the electrical stimulation is separate from the current applied to the needles to release ions, for example, as described with reference to the section “5-ALPHA-REDUCTASE INHIBITOR DEPOSITION”. Optionally, electrical stimulation is applied by one or more discs and/or needles, and ion deposition is applied by different discs and/or needles. Optionally, the needles to apply electrical stimulation but not ion deposition are inert, for example, made from platinum. Alternatively, a voltage is applied to the needles to prevent ion deposition by the galvanic effect. Alternatively or additionally, electrical stimulation and ion deposition overlap, for example, applied by the same discs and/or needles.

Inventors hypothesize that selectively applying a plurality of electrical stimulation patterns (e.g., voltages and/or currents) to the scalp will promote hair growth. However, the efficacy of some embodiments of the invention can be unrelated to the underlying theory, and work even if the theory is incorrect.

In an exemplary embodiment of the invention, the electrical stimulation protocol comprises one or more variables. Non-limiting examples of selectable parameters include:
Geometric voltage and/or current distribution pattern: The pattern of applied voltages and/or current per needle. For example, the voltage and/or current at each needle is independently controlled and/or groups of needles have similar voltages and/or current (e.g., alternating needles have similar voltages and/or currents, needles having the same type of material (for example zinc or copper) have similar voltages and/or currents).
   In some embodiments of the invention, the voltage and/or current pattern is substantially the same, for example, the same needle is associated with the same charge and/or current. Alternatively or additionally, the voltage and/or current pattern is dynamic, for example dynamic throughout the array, and/or a region of the array. For example, in a relatively large array, a relatively small patch of the electrical pattern can be scanned across the array.
   A potential advantage of two groups of needles with different voltages is the controlled patterning of current and/or ion deposition. For example, local stimulation may be superior to global. Potentially, division to several groups allows greater flexibility and/or controllability of the current. For example, current can be applied (e.g., to different groups, at different intensities) simultaneously or in a time-divided manner.
Voltage and/or current distribution pattern over time: The pattern of applied voltage and/or current per needle can vary over time. For example, an alternating current and/or voltage can be applied to vary the voltage and/or current between two or more needles (or groups of needles). In the case of using the device with discs for example in FIG. 1 (e.g., rolling the discs with needles on the scalp), selecting an alternating frequency that is less than the frequency of rotation can result in increasing the diversity and/or gradients of voltages and/or currents applied underneath the skin surface. Inventors hypothesize that applying various patterns of voltage and currents to the skin stimulates hair growth. Potentially, applying varying time and/or location stimulations improves stimulation of local points, for example hair follicles
Direct current (DC) offset: A voltage offset can be applied to the pattern applied to one or more needles. In an exemplary embodiment of the invention, the DC offset is calibrated, for example, from −3 volts to +3 volts, or other smaller, intermediate or larger values are used. In an exemplary embodiment of the invention, the DC disc to disc relative voltage ranges, for example, from 0 to 6 volt, or other smaller, intermediate or larger values are used.
Alternating current (AC) peak to peak voltage: In an exemplary embodiment of the invention, the peak to peak voltage of the AC varies, for example, from −3 volts to +3 volts, or other smaller, intermediate or larger values are used.
Frequency of AC: In an exemplary embodiment of the invention, the frequency of AC ranges, for example, from 10-1000 Hz, or other smaller intermediate or larger values are used.
Waveform of AC: In an exemplary of the invention, the waveform of AC is rectangular. Alternatively, other waveforms are used, non-limiting examples include sinusoidal, triangular, sawtooth.
Current: In an exemplary embodiment of the invention, the total electrical current is less, for example, than 0.5, less than 1, less than 2 milliAmperes, or other smaller, intermediate or larger values are used.

### Adjuvant Treatment

In an exemplary embodiment of the invention, the scalp is stimulated by applying one or more adjuvant treatments. Optionally, at least one drug is applied to the skin.

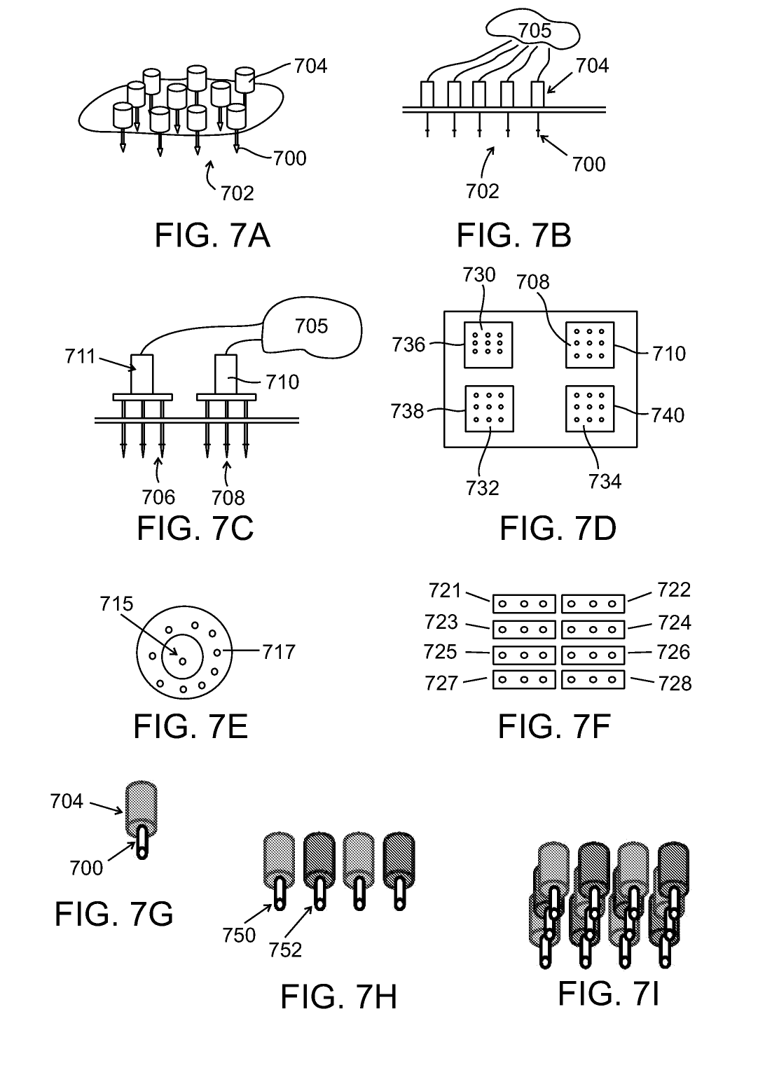

FIG. 9 is an illustration of the device of FIG. 1 further comprising a mechanism for delivering drugs to the skin, in accordance with some embodiments of the invention.

In some embodiments of the invention, at least one drug dispensing mechanism deposits drugs to the skin (e.g., to the surface of the skin, below the surface of the skin).

In some embodiments of the invention, a sponge 902 is used as the drug dispensing mechanism. Optionally, sponge 902 is soaked with the drug to apply to the skin.

In some embodiments of the invention, sponge 902 is in contact with scalp 904. Optionally, sponge 902 is disc shaped, for example having dimensions similar to discs 104. Optionally, sponge 902 is connected to axis 112 similar to discs 104. Optionally, sponge 902 is rolled over scalp 904 together with discs 104. Optionally, sponge 902 is located between discs 104. Optionally, sponge 902 is located between needles. Optionally, sponge 902 is located between discs 104 and needles.

In some embodiments of the invention, two or more sponges are used. Each sponge contains an inactive form of the drug. When the sponges release their drugs into the skin, the drugs react in the skin. A non-limiting example includes one sponge applying a sodium bicarbonate solution to the skin, and another sponge applying an acid for example lemon juice to the skin. The acid and base react in the scalp. Potentially, the reaction of the two solutions pushes out oil and/or fat from the scalp, thereby stimulating the scalp. For example, tiny salt crystals would absorb oil onto their surface. For example, the salt wicks out moisture due to the hypertonicity of the out layer, the wicking also involving flow of sebum and oil.

Non-limiting examples of drugs that can be applied to the scalp (e.g., directly onto the scalp, indirectly by oral administration) include; Minoxidil, Finasteride, Dutasteride, saw palmetto oil.

### Apply Treatment

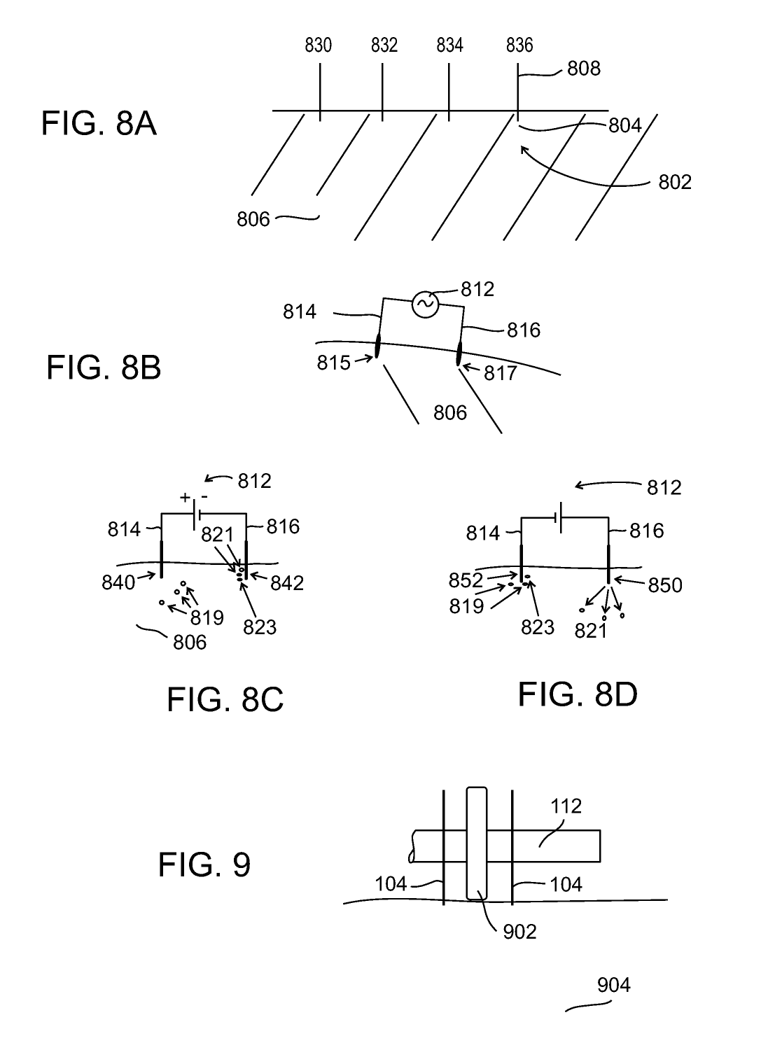

FIGS. 10A-10G illustrate some embodiments of the hair stimulation device. The embodiments are used to apply one or more treatment protocols as described herein to the scalp of the user.

FIG. 10A illustrates a hand held embodiment of the hair stimulation device, in accordance with some embodiments of the invention. A user 1000 holds a hair stimulation device 1002 (e.g., as described with reference to FIGS. 1, 3A and/or 3B) to a scalp 1004.

In an exemplary embodiment, device 1002 comprises a needle array 1006 of discs (e.g., as described with reference to FIG. 1). Optionally, device 1002 is connected to control box 1007. Optionally, control box 1007 comprises a display. Optionally, control box 1007 is connected to power supply 1009.

FIG. 10F illustrates a handleless 1030 version of the hair stimulation device, for example, device 100 without handle 114 as illustrated in FIG. 1. Optionally, user 1000 holds and/or displaces device 1030 across scalp 1004 by holding device 1030 directly at a housing, for example, at housing 110. Optionally, device 1030 is rolled over scalp. Potentially, device 1030 may be easier to move and/or position by some users.

In some embodiments of the invention, user 1000 applies treatment by manually displacing device 1002 with respect to scalp 1004. Optionally, device 1002 applies treatment by rolling over scalp 1004, for example when a needle array 1006 of discs (e.g., as described with reference to FIG. 1) is used. Alternatively or additionally, device 1002 applies treatment when device 1002 has been positioned over the treatment area of scalp 1004, for example by insertion and/or retraction of needles into the scalp, for example, when using array 1006 of needles for example described with reference to FIG. 7A.

In some embodiments of the invention, there is a setting that allows user 1000 to tell device 1002 the area of scalp 1004 being treated. For example, a button or a sensor signals the position to the controller. Optionally, device 1002 changes treatment parameters based on the area of scalp 1004 being treated. For example, areas with relatively less hair may require relatively more intense treatment than areas with relatively more hair.

In some embodiments of the invention, the needle array head of the hair stimulation device is automatically displaced relative to scalp 1004 to apply treatment, for example, by a robot.

FIG. 10D illustrates a robot 1010 moving a needle head array 1008 across scalp 1004 along one axis (e.g., forward or reverse), in accordance with some embodiments of the invention. Optionally, the relative position is known by first calibrating the position of array 1008, for example, by pressing a ‘start’ button.

FIG. 10C illustrates a robot 1012 moving needle head array 1008 over scalp 1004 along two axes, in accordance with some embodiments of the invention.

FIG. 10E illustrates a robot 1014 moving needle head array 1008 over scalp 1004 in a rotational manner, in accordance with some embodiments of the invention. Optionally, robot 1014 comprises motorized spinner 1040.

In some embodiments of the invention, the needle head array of the hair stimulation device is static relative to scalp 1004. Treatment is applied by selectively activating needles, for example insertion and retraction of the needle into scalp 1004, for example, using actuators as described with reference to FIG. 7A.

FIG. 10B illustrates an array of needles 1014 statically positioned over the treatment area of scalp 1004, in accordance with some embodiments of the invention. Optionally, array 1014 covers the entire area to be treated. Alternatively, array 1014 does not cover the entire area to be treated. Optionally, the user moves array 1014 from one location to another. At each location, array 1014 is statically positioned relative to scalp. For example, array 1014 may treat the area around the temple and then move to the area of the vortex.

In some embodiments, the device is intended for one time use. For example, the device may be in the form of a disposable pad with needles, applied manually to the scalp and incorporating a power source, for example, to provide power to deposit ions. Alternatively, no independent power source is necessary. For example, the device relies on galvanic reactions for power. Optionally, the device may be used a limited number of times, for example 5, 10 or 30 times. Optionally, the limited use of the disposable and/or limited use pad provides greater sterility and/or decreases the change of infection. Alternatively, the pad is disposable but connects to a reusable power source.

FIG. 10G illustrates a helmet 1016 comprising needles, positioned at least over some of scalp 1004, in accordance with some embodiments of the invention. Needles are selectively activated to treat regions of scalp 1004, for example, selectively inserted and retracted. Optionally, helmet 1016 remains statically positioned relative to scalp 1004. Optionally, helmet 1016 comprises chin strap 1042. In an exemplary embodiment of the invention, helmet 1016 treats all areas of scalp 1004. Optionally or alternatively, helmet 1016 treats all areas of scalp 1004 automatically.

### Controller

In an exemplary embodiment of the invention, a controller (e.g., controller 308 as described with reference to FIG. 2) controls the application of one or more treatment protocols (e.g., mechanical stimulation, vibration, heat, ion discharge, drug delivery, electrical stimulation, as described herein) by the hair stimulation device. Optionally, the controller is integrated with the device itself, for example logic embedded in the body of the device (e.g., in housing 110 and/or handle 114 as described with reference to FIG. 1). Alternatively or additionally, the device is connected to a separate control box housing the controller. Alternatively or additionally, the device and/or the control box are connected to a computer, having control software thereon.

In an exemplary embodiment of the invention, the controller performs monitoring functions of the hair stimulation device, for example during application of treatment, for example, as will be described in the section “FEEDBACK/MONITORING”.

In an exemplary embodiment of the invention, the controller collects feedback data of the treatment by the hair stimulation device, for example as will be described in the section “FEEDBACK/MONITORING”.

In an exemplary embodiment of the invention, the treatment is administered by controller 308 according to logic (e.g., a software module), for example using a table. Optionally, the table is stored on a memory (e.g., memory 306 as described with reference to FIG. 2). In an exemplary embodiment of the invention, the table contains treatment parameters correlated with values, non-limiting examples include; patient age, degree of baldness, effectiveness of previous treatment parameters. Optionally, the treatment parameters are based on trial and error, for example, empirical data collected from the patient for example by sensor 304 and/or controller 308 during the treatment. Alternatively or additionally, treatment parameters are based on experimental data from a population of subjects. Alternatively or additionally, controller 308 is manually programmed, for example, by a physician. Alternatively or additionally, controller 308 operates according to mathematical models (e.g., equations).

### Feedback/Monitoring

In some embodiments of the invention, the application of treatment for example using one or more treatment protocols is monitored. Optionally, monitoring comprises comparing the delivered treatment against the selected protocol.

In some embodiments of the invention, feedback regarding the application of treatment for example using one or more treatment protocols is obtained. Optionally, feedback is analyzed to estimate the effectiveness of treatment using the selected protocol.

In some embodiments of the invention, hair growth is measured. Optionally, hair growth is measured manually, for example, by taking a picture of the scalp (the entire scalp and/or a zoom-in of a particular area) and having an expert provide an assessment, for example, by counting the number of hairs. Alternatively or additionally, software automatically analyses the pictures, for example, counting the hairs.

In some embodiments of the invention, monitoring and/or feedback is dynamic, for example, to evaluate the application of the protocol during the treatment. Alternatively or additionally, feedback and/or monitoring occurs over a substantially long period of time, for example, to evaluate the application of the protocol over several treatment sessions.

In some embodiments of the invention, one or more sensors, for example sensor 304 described with reference to FIG. 2 are utilized in monitoring and/or feedback of treatment.

In some embodiments of the invention, impedance of the scalp is measured. A potential usage is to monitor the piercing of the skin of the scalp by the needles. Optionally, impedance is evaluated for the entire needle array. Alternatively or additionally, impedance is evaluated for groups of needles and/or individual needles.

In some embodiments of the invention, relatively high impedance is significant, for example, suggesting a lack of sufficient contact for example between the needles and the scalp. Non-limiting examples of relatively high impedance include over 10⁵ ohm, over 10⁶ ohm, over 10⁷ ohm, or other smaller, intermediate or larger values are used. Another non-limiting example is the currently measured impedance relative to previously measured impedance (e.g., of the same patient), for example, over 5×, 10×, 25× higher, or other smaller, intermediate or larger measurements are used.

In some embodiments of the invention, relatively low impedance is significant, for example, suggesting a short circuit, for example between the needles due to an excessively sweaty scalp. Non-limiting examples of relatively low impedance include, for example, less than 10² ohms, less than 10³ ohms, less than 10⁴ ohms, or other smaller, intermediate or larger values are used. Another non-limiting example is the currently measured impedance relative to previously measure impedance (e.g., of the same patient), for example 0.1%, 1%, 10% of the previous value, or other smaller, intermediate or larger measurements are used.

In some embodiments of the invention, the number of needle pricks applied to the skin is calculated and/or estimated. Optionally, the number of disc rolls (and partial rolls) is counted

In some embodiment of the invention, the position of the needle array relative to the scalp is estimated, for example the location of the needles piercing the skin. Optionally, the position is compared to evaluate if the user and/or robot treated the selected areas of the scalp. For example, the position is evaluated using a sensor, for example, an accelerometer. In a non-limiting example, position tracking is measured relative to a starting position. The accelerometer measures acceleration, which is integrated over time to calculate distance. The initial position and angle relative to the scalp are used to create a movement map.

In some embodiments of the invention, the skin temperature is estimated, for example by a temperature sensor. Alternatively or additionally, the change of color of skin is estimated, for example by an optical sensor. A potential advantage is to obtain feedback about the extent of treatment. For example, an increase in skin temperature and/or change of skin color (e.g., reddening) can signify the onset of the inflammatory response in response to the treatment, possibly signifying that the area of skin has been sufficiently treated.

In some embodiments of the invention, monitoring and/or feedback are manual, for example, a physician or other trained practitioner observing the user using the device and/or providing comments to the user. For example, a physician or other trained practitioner visually examining the skin scalp of the user, for example for hair growth, for example, between treatment sessions.

In some embodiments of the invention, monitoring and/or feedback is provided for the user. Optionally, monitoring and/or feedback are subjective. Non-limiting examples include; the degree of pain experienced during treatment, user perception of effectiveness of hair stimulation, user compliance with the protocol.

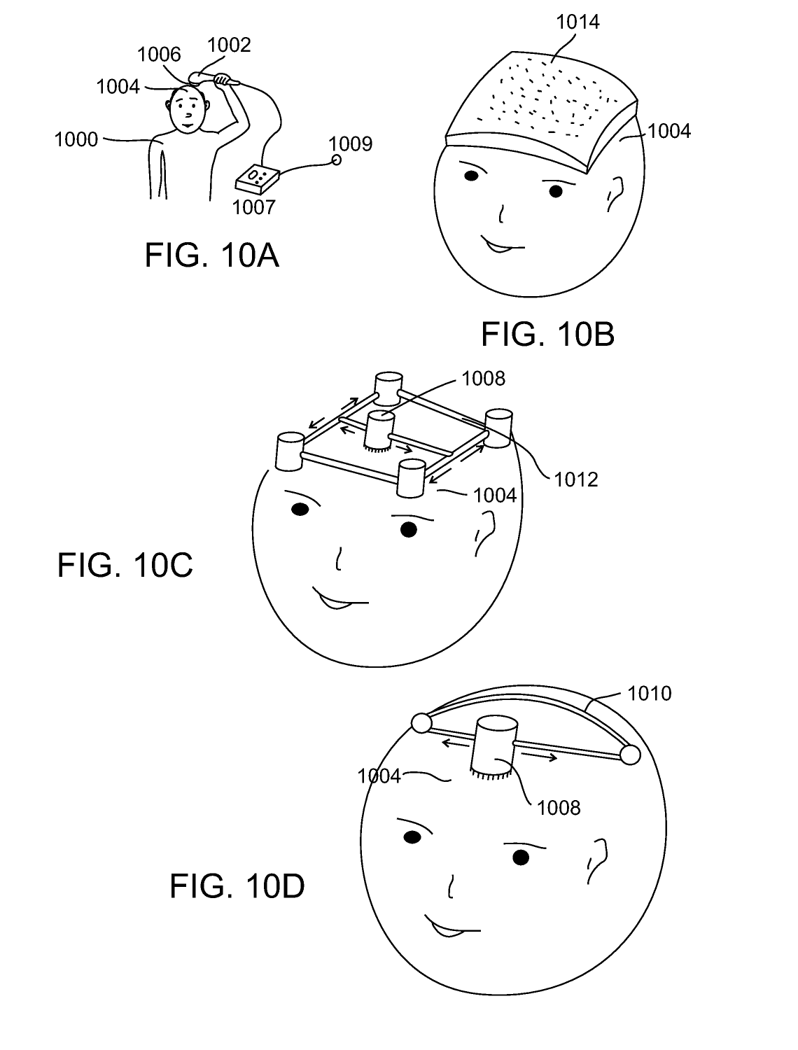

FIG. 11 illustrates a monitoring and/or feedback set-up used with the hair stimulation device, in accordance with some embodiments of the invention. Optionally, a control box 1102 comprises one or more user interfaces 1104 displaying information about the use of the device. Alternatively or additionally, a computer 1106 (containing control software thereon) displays monitoring and/or feedback information about the use of the device on a monitor.

In some embodiments of the invention, the monitoring and/or feedback data is sent to a remote location, for example, to be analyzed by a trained professional.

In some embodiments of the invention, the device, control box 1102 and/or computer 1106 connect to the internet to download and/or upload data. Connections can be wired and/or wireless.

### Adjust Treatment Parameters

In some embodiments of the invention, one or more treatment parameters of the treatment protocols are adjusted. Optionally, adjustments are made according to monitoring data. Alternatively or additionally, adjustments are made according to evaluation data. Alternatively or additionally, adjustments are made according to predetermined changes in the treatment parameters of the treatment protocols, for example over time and/or over treatment sessions.

In some embodiments of the invention, adjustments are made dynamically, for example during the treatment session.

In some embodiments of the invention, adjustments are made before the start of the next treatment session.

In some embodiments of the invention, the intensity of treatment is relatively increased. For example, if the treatment is not sufficiently effective. Non-limiting examples include; the addition of another stimulation protocol (e.g., adding thermal stimulation), relatively increased density of needle pricks, relatively increased dose of ions deposited.

In some embodiments of the invention, the intensity of treatment is relatively reduced. For example, if the treatment is causing side effects, for example pain, excessive swelling and/or bleeding. Non-limiting examples include; removing a stimulation protocol (e.g., removing thermal stimulation), relatively thinner needles, relatively reduced density of needle pricks, relatively reduced dose of ions deposited.

### Repeat

In an exemplary embodiment of the invention, treatment is repeated.

In some embodiments of the invention, treatment is repeated once the skin has sufficiently healed, for example, when the inflammatory response has subsided.

In some embodiments of the invention, treatment is repeated according to the treatment protocol, for example, once every 3 days, or once every 5 days, every 7 days, or other smaller, intermediate or larger time frames are used.

### Kit

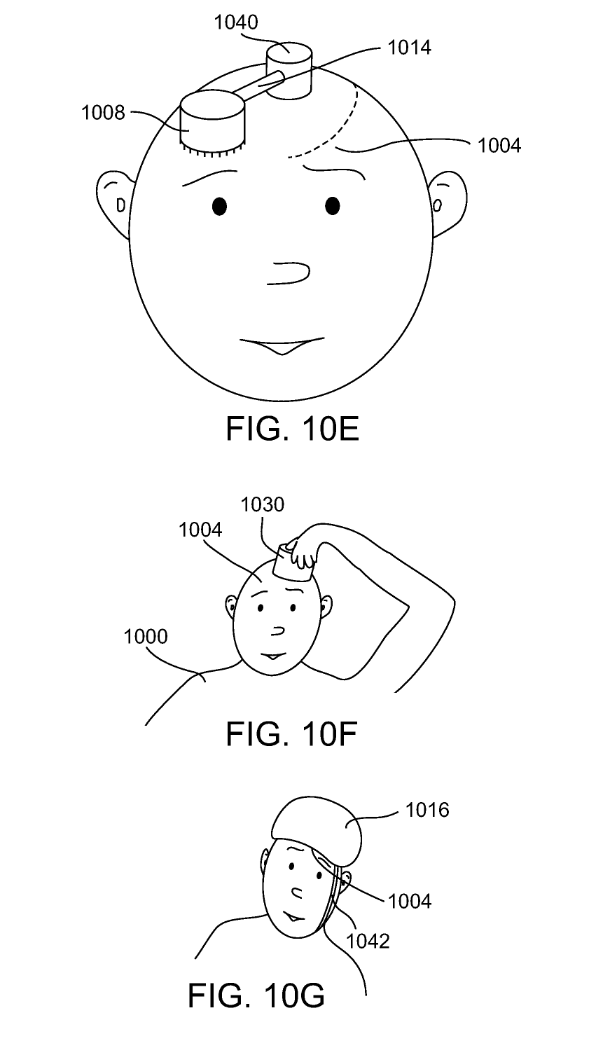

FIG. 12 illustrates components of a hair stimulation kit 1200 in accordance with an exemplary embodiment of the invention. One or more parts of kit 1200 can be sold separately or as a package.

In some embodiments of the invention, kit 1200 comprises a hand-held hair stimulation device 1202. Optionally, device 1202 comprises batteries, for example rechargeable batteries.

In some embodiments of the invention, kit 1200 comprises control box 1204. Box 1204 can be used to perform one or more controller functions for device 1202.

In some embodiments of the invention, kit 1200 comprises an AC adapter 1206. Adapter 1206 has a plug 1208 to fit a standard electrical outlet (for example in a home), and a connector 1210 to fit device 1202.

In some embodiments of the invention, kit 1200 comprises one or more disc replacement packages 1212. Alternatively or additionally, discs 1212 are sold separately. Discs 1212 can come in a variety of designs, for example depending on the selected treatment protocols. One or more non-limiting examples of variations in discs 1212 include; number of discs per package, diameter of discs, size of needles on discs, number of needles per disc, spacing between discs, metallic coating, drug delivery sponge.

Some non-limiting examples of disc variations include;

4 discs (e.g., instead of 8), for example, for those with long or dense hair, the 4 discs having double the space between the discs relative to the 8 disc arrangement.

Needles having a pricking depth ranging from 0.1 mm to 1 mm pricking depth (e.g., 0.2 mm, 0.4 mm, 0.6 mm, 0.8 mm).

Discs with additional 5 or 10 mm in diameter; for example, for long or dense hair.

Discs coated with Zn, Cu, Fe, or no coating.

In some embodiments of the invention, discs 1212 are disposable. Alternatively or additionally, discs 1212 can be sterilized.

In some embodiments of the invention, kit 1200 comprises one or more drugs 1214, for example topical additives. Alternatively or additionally, drugs 1214 are sold separately. Non-limiting examples of additives include; Soothing gel: 60 to 120 ml; minoxidil solutions: 30 to 60 ml; other non-drug hair promoting (e.g. sow palmetto oil): 60 to 120 ml; hair strengthening shampoo: 100 to 500 ml.

In some embodiments of the invention, kit 1200 comprises cleansing wipes and/or other cleaning aids, for example shampoo.

Some non-limiting examples of exemplary kits include;
Device 1202, control box 1204 and AC adapter 1208.
Disc replacement 1212, topical additive 1214.
Device 1202, control box 1204 and AC adapter 1208, disc replacement 1212.
Device 1202, control box 1204 and AC adapter 1208, disc replacement 1212, additive 1214.

### Light Stimulation

In an exemplary embodiment of the invention, light (visible or infrared) is applied to the skin, either below the scalp surface, for example, by means of internal light sources or guides, or from above the surface. Optionally, light guides are transparent discs or needles or optical fibers embedded in the discs or needles. Optionally, the optical fiber replaces the needles. Optionally, the light source is located at the axis of the wheel.

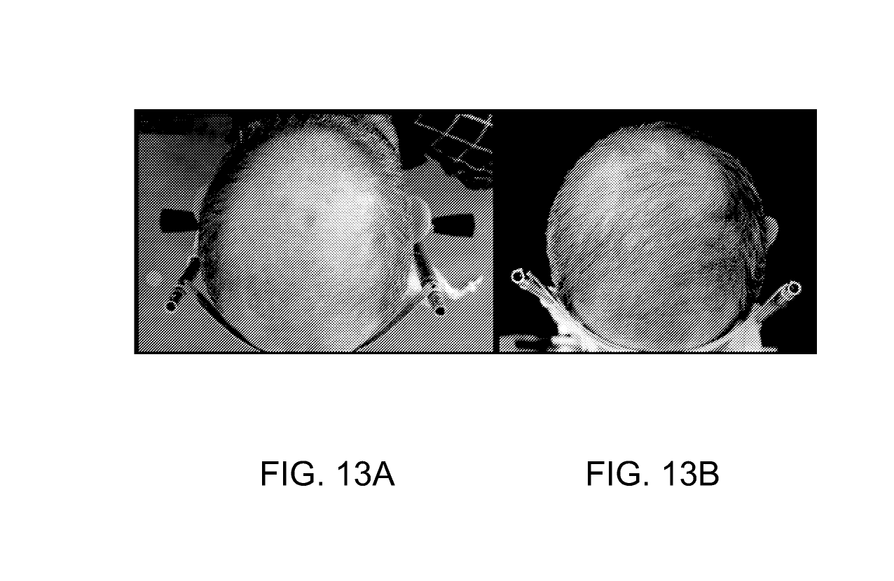

FIGS. 14A and 14B illustrate discs comprising a light source. In an exemplary embodiment, light conducting disc 1400 (FIG. 14A) comprises light source 1402 causing light 1404 to emanate from spike 1410 on disc 1400. Optionally, disc 1400 comprises translucent material. Optionally or alternatively, spike 1410 comes to a sharp point. Optionally, spike 1410 is metallic.

FIG. 14B illustrates an exemplary embodiment in which light 1404 originates from light source 1402 and travels through optical fibers 1406 embedded in disc 1400. Optionally, the optical fibers 1406 penetrate directly into the skin. Optionally, optical fibers 1406 are thin enough to easily penetrate skin.

In some embodiments, one or more discs each comprise multiple fibers and/or needles. Optionally, at least one disc is for optical stimulation. Optionally or alternatively, at least one disc is metallic. Optionally or alternatively, at least one disc includes both optical needles and metallic needles. Optionally, at least one needle is both optical and metallic. Optionally or alternatively, fiber and/or needle are provided on parallel discs. Optionally or alternatively, fibers and/or needles are provided in a planar array.

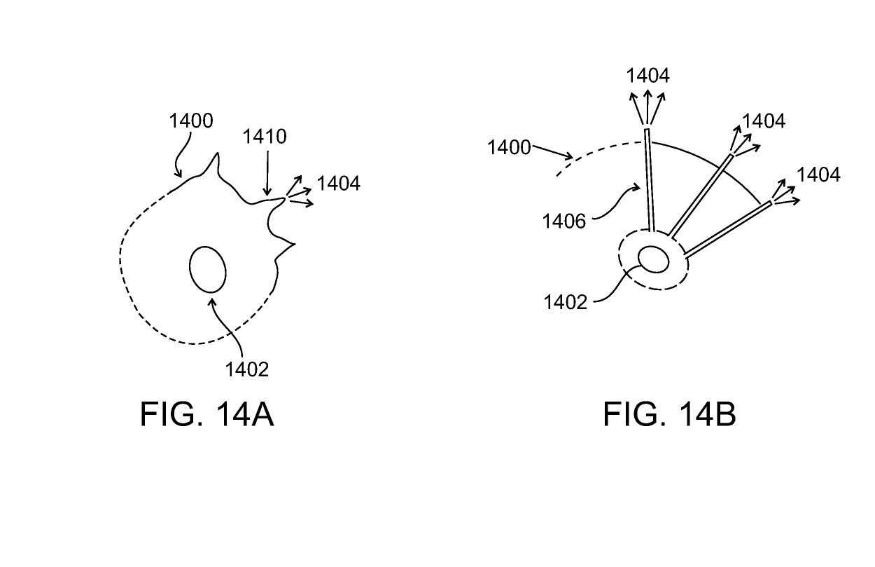

FIG. 15 illustrates an injector comprising a light guide, in accordance with an exemplary embodiment of the invention. In an exemplary embodiment, the light guide is an optical fiber coated with metal. For example, light is produced by light source 1502 which is powered by power source 1500 and emanates light 1504. Optionally, power source 1500 is electrical.

In some embodiments, power source 1500 emits ions 1508 directly into the scalp beneath the scalp surface 1506. Optionally, power source 1500 emits electricity directly into the scalp beneath the scalp surface 1506. Optionally, power source 1500 emits heat directly into the scalp beneath the scalp surface 1506. Optionally, the discs, needles and/or optical fibers also vibrate.

In some embodiments, the injector comprises a cavity 1512. Optionally, cavity 1512 comprises a light conducting core. For example, cavity 1512 may comprise light transmitting material. Optionally, the light transmitting material has structural rigidity. Optionally or alternatively, the light transmitting material has minimal structural rigidity.

In some embodiments, cavity 1512 comprises an internal optical fiber. For example, the internal optical fiber may comprise a metal coated thin optical fiber. Optionally or alternatively, the internal optical fiber may comprise an external shell conducting electricity. Optionally or alternatively, the internal optical fiber may comprise an external shell conducting heat. Optionally or alternatively, the internal optical fiber may comprise an external shell conducting injecting ions into the skin. Optionally or alternatively, the internal optical fiber may emit light into the skin.

In some embodiments, hollow cavity 1512 comprises a void which transmits light. Optionally, the outer portion 1510, inside outer layer 1512, of the injector comprises a source of vibration. Optionally or alternatively, the outer portion of the injector comprises a source of heat.

In some embodiments, the outer layer 1514 comprises an electrical conductor. For example, outer layer 1514 comprises metal. Optionally, outer layer 1514 is coated with ions to be deposited. For example, outer layer 1514 is coated with Cu. Alternatively, outer layer 1514 is coated with Zn. Optionally, outer layer 1514 comprises heat conducting material.

Various embodiments and aspects of the present invention as delineated hereinabove and/or as claimed in the claims section below find experimental support in the following examples:

### EXAMPLE

### Experiment

Reference is now made to the following example, which together with the above descriptions illustrates some embodiments of the invention in a non-limiting fashion. In particular, features described below may be used without other described features and in conjunction with methods and/or apparatus as described above.

Patient Profile:

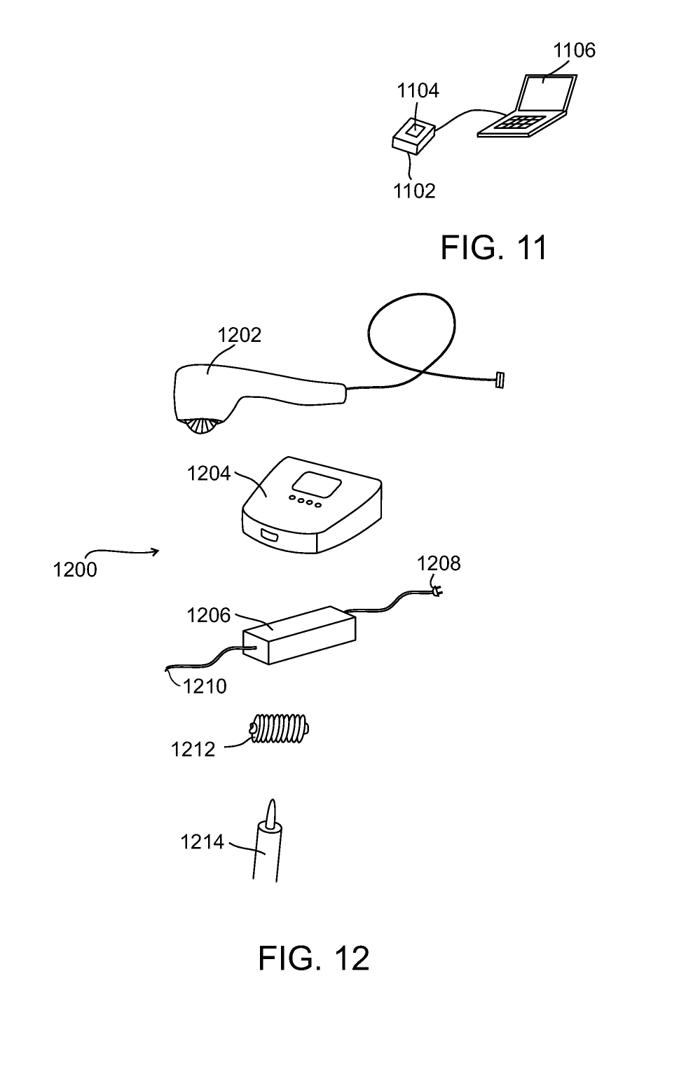

The subject of the experiment was one of the inventors, Mr. Dov Ingman. Mr. Ingman is 63 years old. He has been slowly balding for the past 30 years. FIG. 13A is an image of Mr. Ingman's scalp taken during October 2007, before treatment was started on February 2011. The state of hair at the beginning of the treatment was very similar to the one in the 2007 picture]

Treatment Parameters:

Mr. Ingman is a potential candidate for treatment using the hair stimulation device as in some embodiments described herein, for example, satisfying inclusion criteria, and not fitting any exclusion criteria.

Treatment was selected to be applied to the entire scalp, a total treatment area of about 40 000 mm² (e.g., 200 mm×200 mm). The treatment protocol was initially selected to be 2-4 minutes, applied on a daily basis. The treatment regimen results in mild pain. To try and reduce the pain, the treatment parameters were changed to 2-3 sessions per week, for about 5 minutes per treatment and at a reduced vibration amplitude. The change resulted in reducing the pain to a tolerable level of subtle pain. Furthermore, the frequency and/or time duration have been selected to achieve a balance between sufficient stimulation and sufficient recovery of the skin from the stimulation (e.g., to withstand another treatment session).

Device Selection:

The device used was the hand-held version of the hair stimulation device, as illustrated in FIG. 3A. The device uses discs with needles on the circumference of the discs, as illustrated in FIG. 3C. The design of 8 discs has been selected. Alternating discs are coated with copper and/or zinc. The radius of each disc is 16 mm. Each needle on the disc is 1 mm apart. The area that each needle pricks is about 0.0001 mm² (e.g., using square needles of 0.1 mm×0.1 mm). Discs are 3 mm apart.

Mechanical Stimulation Protocol:
The density of pricks has been prescribed at about 10 pricks per mm² of the scalp. Examples of parameters to achieve this density:
With a circumference of about 100 mm (radius of 16 mm) and a needle spacing of 1 mm, one roll of the disc will prick the scalp 100 times. 8 discs, rolled 500 times over the scalp (in a single treatment session) will prick the scalp about 400 000 times.
The total area of all the pricks during the treatment session is about 40 mm². The percentage of the total area of the scalp that is treated per session is about 0.10%.

Vibration Protocol:
The vibration of the needles has been selected to be about 50 Hz, with vibrations both perpendicular to the scalp surface and sideways perpendicular to the path of motion.
In an exemplary embodiment of the invention, the area per prick can be increased about 10×, for example, by an omni-directional vibration of the needles (e.g., by vibrating the discs). A potential advantage is to increase the mechanical stimulation if the current parameters are evaluated as not being sufficiently effective.

Ion Deposition Protocol:
The density of type I alpha-reductase inhibitors (zinc and copper) has been selected to be about 8 nano-grams per cm² of skin for each of the ion types. Examples of parameters to achieve this density:
During one treatment, a total charge of 100 nano-Amperes per second through all of the zinc coated needles, and a similar charge through all of the copper coated needles, will result in a deposition density of over 8 nano-grams per cm² of skin for each of the ion type. (Atomic weight of zinc is 65.4 gram/mol, atomic weight of copper is 63.5 gram/mol, oxidation state of zinc and copper is 2.)

Thermal Stimulation Protocol:
The heat applied to the tissues has been selected to result in a tissue temperature in the range of 50-60 degrees Celsius.

Electrical Stimulation Protocol
The voltage applied between the discs coated with Zinc and the discs coated with Copper has been selected to be an alternating current (AC) at a frequency of 100 Hz, with a peak to peak voltage of 5 V. The current has been selected not to exceed 2 mA.

### Results

FIG. 13B is an image of taken during September 2011. The increase in the amount of hair in comparison to FIG. 13A is significant. The increase in the amount/density of hair seems to vary among different regions. There seem to be more active follicles, with significant conversion from vellus hair to terminal hair. The treatment was applied to all balding areas. The impression provided is an expert's opinion. The expert was closely monitoring the progress.

### ADDITIONAL EXAMPLE

### Experiment

Additional experimental results suggest that the more a person uses the device, the quicker and/or more complete the restoration, enhancement and/or causation of hair growth. Results were improved when treatment was daily and when treatment was focused during a session on a smaller area. Specifically, results were apparently improved for use of the device on smaller area for less time as compared with use of the device on a larger area for more time.

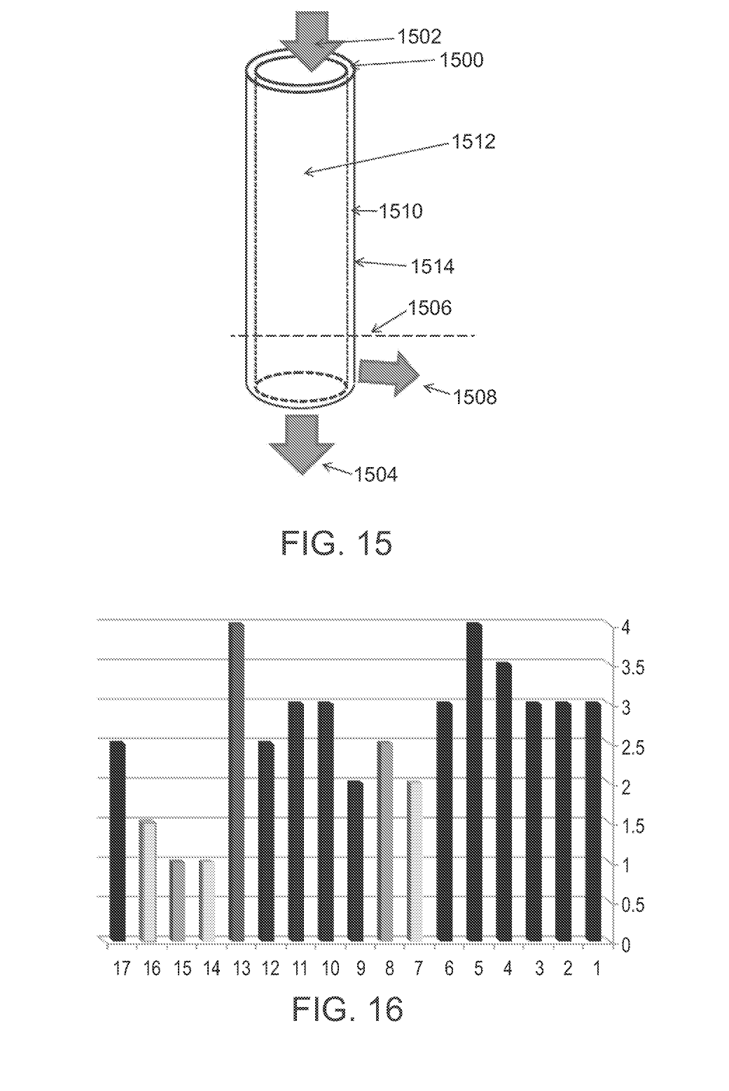

For example, FIG. 16 is a bar chart summarizing interim results of treatment on subjects who were treated with the device. Overall, 96 patients appeared for the study, with an average age of 40 (89 men, comprising 93% of the sample, with an average age of 39, and 7 women, comprising 7% of the sample, with an average age of 55). 8 patients (8% of the sample), all male, with an average age of 46, did not receive a device because they were rejected from the study or were not interested in receiving the device. 88 patients received a device (Second Generations Pilogics Apparatus for Stimulating Hair Growth and/or Preventing Hair Loss) in total. 21 patients, 20% of the overall sample, all male, with an average age of 43 received a device but left the study before completing 6 months of treatment. 67 patients received a device, with a mean age of 40 (60 men, comprising 80% of patients receiving a device, at an average age of 51, and 7 women, comprising 20% of patients receiving a device, at an average age of 39).

The first 35 patients to receive the device entered the study between 1 Nov. 2011 and 29 Feb. 2012 (30 men and 5 women). Of those 35 patients, 17 men (56% of the 30 men who began treatment), average age of 41, and 4 women (80% of the 5 women who began treatment), average age of 53, completed 6 months or more of treatment with the device. [The age of one female patient, 80, was significantly above average]. 41% of men were age 20-30; 35% of men were age 30-35; and 23% of men were over age 50. 75% of women were age 30-50; 25% of women were over 50.

The bar chart in FIG. 16 summarizes the results of the 17 male subjects who completed at least six months of treatment with the device. Each subject was assigned a score between 0-4, as seen on the y-axis of the bar chart. 0 indicates that a patient withdrew from treatment before the completion of 6 months of treatment. 1 indicates no improvement from the beginning to the end of treatment, as seen in a comparison of the first and last photograph taken of the scalp of the subject. 2 indicates a slight improvement seen between the first and last photo. 3 indicates a tangible improvement seen between the first and last photo. 4 indicates a significant improvement between the first and last photo. Each patient who completed treatment was assigned a bar reaching a height of between 1 and 4, depending on their results.

Each patient was further assigned a color to their bar depending on the initial state of their hair loss. Subjects who began the treatment with a slight amount of hair on their scalp were assigned a black bar. Subjects with patches of baldness were assigned a gray bar. Subjects with large areas of baldness were assigned a white bar.

As reflected in the bar chart, 2 of the 17 male patients saw no improvement; 1 patient saw an improvement described as intermediate between no improvement and minor improvement, 2 patients saw minor improvement, 3 patients saw improvement described as intermediate between minor improvement and tangible improvement, indicates a significant improvement; 6 patients saw tangible improvement; 1 patient saw improvement described as intermediate between tangible improvement and significant improvement; and 2 patients saw significant improvement.

Of the 4 women who began treatment with the device, 3 completed at least 6 months of treatment. All 3 women who completed the treatment saw tangible improvement (a score of 3 if they were to be included in the bar chart).

None of the patients who used the device saw their hair recede during treatment.

### Treatment Protocol

In accordance with the protocol of the study, patients received an average of 2-3 treatments a week for five minutes. Each patient received a Prologics Generation 2 device and a frequency of treatment of 5 minutes 3 times per week in the first month. According to the protocol, patients were permitted to increase the frequency of treatment.

### Results

1. Frequency of treatment: Men were found to be more likely to choose the option of increasing the frequency of treatment. Additionally, several patients failed to follow the prescribed frequency of treatment, for various reasons. Overall, diminished results were seen for those patients who received less than the prescribed treatment.
2. None of the patients who used the device saw their hair recede during treatment.
3. 53% of patients had improvement evaluated as between 3-4, according to the 0-4 evaluation scale described above, indicating tangible to significant improvement. The average age of the three patients receiving significant improvement (score 3.5 to 4) was 46 (53, 33 and 52). Three with the rank of alopecia hair medium short writers, and hereditary baldness. The 33-year-old patient received notable improvement, with a very significant change observed from test to test. That patient received treatment diligently every day for approximately 20 minutes. His hair is on the way back to full coverage.
4. Of the patients between age 50 and 59, they each began treatment with short hair and with the same moderate degree of baldness, spread out over the entire upper area of the scalp and seen more at the top. These patients underwent continuous treatment, with 80-100% conformity with the protocol.
5. 6 patients (35% of the patients) received scores of 3, indicating tangible improvement. These patients saw and felt their hair improving and the photographs also show a positive visual change. Two of these patients (33% of the patients) were between age 50 and age 59, and 66% of these patients were between age 20 and age 30. The majority of these patients began treatment with a moderate degree of baldness spread out over the entire upper area of the scalp and seen more at the top (as with the group which saw significant improvement). It is possible to conclude that men around the age of 50 with a moderate degree of baldness spread out over the entire upper area of the scalp have a high potential to improve significantly than younger people who have the same degree and location of baldness. The youngest patient in this group, 23 year old patient, received an additional point because, in his case, baldness began at a very young age and the receding of his hairline occurred very quickly and there was a significant change in relation to what would have been without treatment.
6. 3 patients (17% of the patients) received a score of 2.5, indicating an improvement that the patient feels and possibly sees, but is still not particularly striking, and can principally be seen in photographs.
7. 5 patients (29% of the patients) received disappointing results in relation to the others, receiving scores between 1 and 2. The majority of these patients were between age 30 and age 39.
8. In all a significant improvement was seen in 18% of the patients; tangible improvement was seen in 35% of the patients; a moderate improvement was seen in 18% of the patients; and a minor improvement or no improvement was seen in 29% of the patients.

To summarize the results, 70% of the male patients who underwent treatment for 6 months or more received results ranked between 2.5 and 4 and self reported that the treatment had a positive effect on their hair, that the treatment brought a cessation to the consistent and sustained, multi-year, gradual decline in the density of their hair and in the area of coverage their hair supplied to their scalp was. The patients further reported a sharp and fast change from receding hair to improving hair density and coverage at a level marked by visual improvement during treatment.

Patients reported feeling positive about the progress of the treatment even when visible change was difficult to detect. Most patients were satisfied with the results they received. No patients reported complaints about pain during treatment.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

It is expected that during the life of a patent maturing from this application many relevant hair stimulation devices will be developed and the scope of the term hair stimulation device is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

### General

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

What is claimed is:

1. A device to stimulate a scalp comprising:
an array of stimulating elements adapted to pierce skin of said scalp no deeper than a thickness of a dermis, said stimulating elements are arranged along a circumference of at least one wheel, said wheel adapted to roll over said scalp.

2. The device of claim 1 wherein said stimulating elements comprise needles.

3. The device of claim 1 or claim 2 wherein said stimulating elements release ions.

4. The device of any one of claims 1-3, wherein said stimulating elements are further adapted to additionally stimulate the scalp to enhance movement of said ions to a level sufficient to cause a significant biological effect.

5. The device of any one of claims 1-4, wherein,
a first group of stimulating elements of said array comprise a first metal;
a second group of stimulating elements of said array comprises a second metal; and
the electrochemical gradient is sufficient to cause ions to travel in an amount sufficient to cause a biological effect.

6. The device of any one of claims 1-4, wherein:
said stimulating elements of said arrays comprise at least one metal; and
said metal has an electro potential sufficient to cause ion injection.

7. The device of any one of claims 3-6, wherein said ions comprise at least one of copper ions and zinc ions.

8. The device of any of claims 1-7, wherein:
said stimulating elements are arranged to part hair on scalp during piercing by said stimulating elements;
said arranged to part hair comprises stimulating elements separated from one another along a dimension to form a gap sufficiently wide to avoid trapping hairs; and
said dimension comprises between 2 mm and 5 mm.

9. The device of any one of claims 1-8, wherein:
said stimulating elements of said array are separated by 0.1 mm to 1 mm along an axis;
said stimulating elements are adapted to pierce skin to at least one depth between 100 micrometers and 1700 micrometers; and
said stimulating elements have a shape that forms a wound with a cross sectional area between 0.00001 mm² and 0.1 mm² at said dermis.

10. The device of any one of claims 1-9, further comprising at least one element to vibrate at least one stimulating element.

11. The device of claim 10, wherein said vibrate comprises vibrating to increase a cross section of a wound under said scalp by said stimulating element by a factor ranging from 2×-20×.

12. The device of claim 10 or claim 11, wherein said vibrate comprises vibrating at a frequency ranging from 50 Hz-120 Hz and at an amplitude of 0.05 mm to 0.2 mm.

13. The device of any one of claims 1-12, wherein at least one stimulating element is electrically coupled to a power source.

14. The device of claim 13, further comprising:
ion injecting electrodes that touch the scalp connected to one terminal of said power source; and
an electrode that does not touch the scalp connected to a second terminal of the power source.

15. The device of any one of claims 1-14, wherein at least one stimulating element is configured to provide heat adapted to maintain a temperature of said stimulating elements during said piercing of said scalp.

16. The device of any one of claims 1-15, further comprising a controller for regulating the application of at least one of: temperature of said stimulating elements, number of said piercings of said skin by said stimulating elements, vibration of said stimulating elements, application of electrical current by said stimulating elements to said skin.

17. The device of claim 16, further comprising:
a memory, said memory coupled to said controller, said memory containing data correlating stimulation parameters with a treatment; and
a sensor, said sensor coupled to said controller, said sensor configured to monitor said piercing of said skin by said stimulating elements.

18. The device of any one of claims 1-17, further comprising a drug reservoir comprising at least one drug to administer to said scalp.

19. The device of any one of claims 1-18, further comprising at least one of a motor and a handle, each configured to displace said array across said scalp.

20. The device of any one of claims 1-19, further comprising an encoder operable to count revolutions or partial revolutions of said wheel.

21. The device of any of claims 1-20, wherein said stimulation comprises light.

22. The device of any of claims 1-21, wherein said light is delivered through at least one of transparent discs, stimulating elements and optical fibers.

23. The device of any of claims 1-22, wherein said stimulating elements comprise optical fibers acting as needles to pierce skin.

24. The device of any one of claims 1-23, wherein said stimulating elements comprise at least one injector configured to deliver stimulation directly into skin without needles.

25. A device to stimulate a scalp comprising an array of stimulating elements adapted to pierce skin of said scalp no deeper than a thickness of a dermis.

26. The device of claim 25, wherein said stimulating elements are independently displaceable along a long axis of said stimulating elements to pierce said scalp in synchronized motion.

27. The device of claim 25 or claim 26, wherein said stimulating elements comprise needles and further comprising:
a power source in electrical communication with at least two of said needles, said power source coupled to apply a voltage across said at least two needles;
a vibrational element coupled to said array, said vibrational element operable to vibrate said needles along at least one axis; and
a heat source thermally coupled to said needles, said heat source operable to raise needles to a temperature sufficient to raise a temperature of a volume of skin to within a range of 45-70 degrees Celsius.

28. The device of any one of claims 25-27, wherein at least one of said needles is coated or made from a first material that discharges zinc ions and at least one of said needles is coated or made from a second material that discharges copper ions.

29. A method of stimulating a scalp comprising:
forming channels at least below an epidermal layer of skin of said scalp;
providing at least one stimulation from inside said channels;
effecting tissue adjacent to said channels; and
controlling said providing.

30. The method of claim 29, wherein stimulating comprises wounding said skin in a non-contiguous pattern.

31. The method of claim 29 or claim 30, wherein said wounding comprises wounding said skin at a density of 5-10 wounds per mm².

32. The method of any one of claims 29-31, wherein said controlling comprises wounding said skin in a period of time ranging from 0.01 seconds to 0.1 seconds per wound, to reduce a pain level.

33. The method of any one of claims 29-32, wherein said wounding comprises sufficiently wounding said skin to induce a wound healing response that regenerates hair follicles.

34. The method of any one of claims 29-33, wherein said wounding comprises selectively wounding at a depth selected according to a stage of baldness.

35. The method of any one of claims 29-34, wherein said stimulation is provided at multiple depths in the epidermis and/or dermis, ranging between 100 micrometers and 1700 micrometers.

36. The method of any one of claims 29-35, wherein said stimulating comprises applying a vibration under said skin.

37. The method of claim 36, wherein said controlling comprises applying a vibration to increase the cross sectional size of a wound under said skin by a factor of 2-20×.

38. The method of any one of claims 29-37, wherein said stimulating comprises applying light under said skin.

39. The method of any one of claims 29-38, wherein said stimulating comprises applying heat sufficiently to induce a wound healing response that increases collagen production.

40. The method of claim 39, wherein said controlling comprises applying sufficient heat to raise a temperature of a volume of skin to within a range of 45-70 degrees Celsius.

41. The method of any one of claims 29-40, wherein said stimulating comprises applying at least one voltage gradient to an area of said skin.

42. The method of any one of claims 29-41, wherein said stimulating comprises applying voltage in an opposite polarity to release copper ions under said skin.

43. The method of any one of claims 29-42, wherein said stimulation comprises forming a galvanic current that releases zinc ions under said skin.

44. The method of any one of claims 29-43, wherein:
said stimulating comprises applying an alternating current to alternate deposition of copper ions and zinc ions under said skin; and
a waveform of said alternating current is selected according to the ratio of the desired deposition of copper ions and zinc ions.

45. The method of any one of claims 29-44, wherein:
said stimulating comprises depositing a selected amount of at least one of copper ions and zinc ions under said skin to inhibit type I 5-alpha-reductase to stimulate hair growth;
said selected amount of zinc ions ranges from 0.001 to 1 nanogram/cm² per treatment;
the maximum total weekly dosage is between 2 nanograms/cm2 and 4 nanograms/cm2; and
said selected amount of said copper ions ranges from 1% to 50% of said selected zinc ion amount.

46. The method of any one of claims 29-45, wherein said controlling comprises:
applying said stimulation according to a position on said scalp; and
adjusting said providing according to hair growth.

47. The method of any one of claims 29-46, wherein said controlling comprises measuring an impedance to determine contact of at least one needle with said skin.

48. The method of any one of claims 29-47, wherein:
a treatment session of said stimulating said scalp is repeated at least once daily; and
said controlling comprises applying said stimulation during a particular treatment session to an area on said scalp smaller than the entire area being treated.

49. The method of any one of claims 29-48, wherein:
hair loss is treated by said stimulation of said scalp; and
said method further comprises comparing said providing to a treatment plan.

50. The method of any one of claims 29-49, wherein said stimulating comprises depositing at least one of copper ions and zinc ions under said skin to:
enrich the scalp with mineral nutrients in an amount sufficient to cause a biological effect; and
create electrical fields.
